SEQUENCE LISTING (12 sequences)

SEQ ID NO: 1 - protein, 21 aa, Artificial Sequence
  Synthetic amino acid sequence
SGGGSGGGSCGGIAYLKQATK

SEQ ID NO: 2 - protein, 21 aa, Artificial Sequence
  Synthetic amino acid sequence
SGGGSGGGSCGGIAYLKQATK

SEQ ID NO: 3 - protein, 16 aa, Artificial Sequence
  Synthetic amino acid sequence
ANERADLIAYLXQATK

SEQ ID NO: 4 - protein, 16 aa, Artificial Sequence
  Synthetic amino acid sequence
ANERADLIAYLKQATK

SEQ ID NO: 5 - protein, 8 aa, Artificial Sequence
  Synthetic amino acid sequence
SIINFEXL

SEQ ID NO: 6 - protein, 21 aa, Artificial Sequence
  Synthetic amino acid sequence
SGGGSGGGSCGGIAYLKQATK

SEQ ID NO: 7 - protein, 21 aa, Artificial Sequence
  Synthetic amino acid sequence
SGGGSGGGSCGGIAYLKQASK

SEQ ID NO: 8 - protein, 22 aa, Artificial Sequence
  Synthetic amino acid sequence
SGGGSGGGSCGGIYFSPANKKL

SEQ ID NO: 9 - protein, 16 aa, Artificial Sequence
  Synthetic amino acid sequence
SIINFEKLGGGGSGCS

SEQ ID NO: 10 - protein, 16 aa, Artificial Sequence
  Synthetic amino acid sequence
SIAAFASLGGGGSGCS

SEQ ID NO: 11 - protein, 8 aa, Artificial Sequence
  Synthetic amino acid sequence
SIINFEKL

SEQ ID NO: 12 - protein, 8 aa, Artificial Sequence
  Synthetic amino acid sequence
SIAAFASL

(19) United States
(12) Patent Application Publication
(10) Pub. No.: US 2014/0120557 A1
(43) Pub. Date: May 1, 2014

(54) Photocrosslinkable Peptide-MHC Complexes for Antigen-Specific T Cells and Methods of Using the Same

(71) Applicant: The Board of Trustees of the Leland Stanford Junior University, Palo Alto, CA (US)

(72) Inventors: Jianming Xie, Stanford, CA (US); Mark M. Davis, Atherton, CA (US)

(21) Appl. No.: 14/044,129

(22) Filed: Oct. 2, 2013

Related U.S. Application Data
(60) Provisional application No. 61/709,800, filed on Oct. 4, 2012.

(51) Int. Cl. G01N 33/569; C07K 14/00

(52) U.S. Cl. CPC G01N 33/56972; C07K 14/001
USPC 435/7.24; 530/350

(57) ABSTRACT

Methods for labeling and/or detecting a T cell according to specificity of an antigen T cell receptor (TCR) are provided. Also provided are monomeric MHC-peptide complexes and kits for crosslinking to a T cell according to specificity of an antigen T cell receptor (TCR). The methods, monomeric MHC-peptide complexes and kits find use in a variety of applications related to the detection and purification of antigen-specific T cells, such as those T cells involved in tumors, infectious diseases and autoimmune diseases.

### GOVERNMENT RIGHTS

This invention is made with Government support under grant No. AI022511 awarded by the National Institutes of Health. The Government has certain rights in this invention.

### INTRODUCTION

Antigen-specific T cells are implicated in tumors, infectious diseases and autoimmune diseases. T cells express antigen receptors (TCRs) that recognize antigenic peptides bound to the major histocompatibility complex (MHC), e.g. on the surface of antigen presenting cells (APC). Since the interaction between TCR and peptide—major histocompatibility complex (pMHC) is generally very weak, and with a rapid dissociation rate, a soluble pMHC monomer does not, in most cases, stably adhere to the T-cell surface. While pMHC tetramers and other multimeric forms (such as dimers, pentamers, dextramers, lipid vesicles, and quantum dots, etc.) are used in both basic and clinical T-cell immunology to detect and isolate specific T cells in mixed populations, there is an interest in providing a more stable interaction.

The half-life of the pMHC-TCR interaction is important for T cell signaling events and T-cell activation. As such, methods and reagents for labeling and detecting antigen-specific T cells with specificity and efficiency are of interest, e.g., in the diagnosis of disease. The present invention addresses this issue.

### SUMMARY OF THE INVENTION

Methods for labeling and/or detecting a T cell according to specificity of the T cell antigen receptor (TCR) are provided. In the methods of the invention, a T cell is contacted with a monomeric MHC-peptide complex comprising a peptide, wherein the peptide comprises a core TCR-recognition region; and a photocrosslinking moiety that is located outside of the core TCR-recognition region of the peptide, under conditions where TCR and the core TCR-recognition region specifically bind; and applying a stimulus to crosslink the MHC-peptide complex to the TCR.

Also provided are monomeric MHC-peptide complexes and kits for crosslinking to a T cell according to specificity of an antigen T cell receptor (TCR). The methods, monomeric MHC-peptide complexes and kits find use in a variety of applications related to the detection and purification of antigen-specific T cells, such as those T cells involved in tumors, infectious diseases and autoimmune diseases.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates the structure and synthesis of representative peptides: (a) synthesis of BioMCC(ASA) a photocrosslinkable derivative of MCC peptide (underlined is the MHC-binding and TCR-recognition region); (b) exchangeable peptide Lys99ANP, a Lys99→ANP mutant of MCC peptide; and (c) exchangeable derivative of MCC peptide (Lys99→β-homoLeu).

FIG. 2 illustrates use of a photocrosslinkable pMHC monomer to specifically and efficiently stain antigen-specific T cells with excellent: (a) 5C.C7 T cell blasts photocrosslinked with BioER60(ASA)-I-Ek, Bio102S(ASA)-I-Ek, and BioMCC(ASA)-I-Ek, respectively; (b) 2B4 T cell blasts photocrosslinked with BioMCC(ASA)-I-Ek in the presence (blue) or absence (green) of anti-2B4-TCR antibody; (c) Listeria-specific T-cell clone photocrosslinked as in (a); (d) AND T cell blasts photocrosslinked with BioMCC(ASA)-I-Ek and BioER60(ASA)-I-Ek, respectively; and (e) Naïve OT-1 T cells photocrosslinked with OVA(ASA)-Kb and the null SIAAFASL(ASA)-Kb.

FIG. 3 shows that T cells do not flux calcium in response to TCRs being covalently bound with soluble pMHC monomers, but do after subsequent aggregation of pMHC-TCR pairs: (a) 5C.C7 T cell blasts loaded with Fura-2 AM dye, photocrosslinked with BioMCC(ASA)-I-Ek, SAv-QD705 was added to T cells before imaging; and (b) time-lapse montage of calcium response and QD image from a representative T cell.

FIG. 4 illustrates that T cells flux calcium and form immunological synapses in response to covalently bound pMHC ligands on planar lipid bilayers: (a) 5C.C7 T cell blasts, loaded with Fura-2 AM dye and photocrosslinked with BioMCC(ASA)-I-Ek, and added to a planar lipid bilayer for calcium imaging; and (b) 5C.C7 T cell blasts photocrosslinked with BioMCC(ASA)-I-Ek, labeled with H57-scFv-A647 conjugates, and then added to a planar lipid bilayer presenting ICAM-1-A555 and B7-1.

FIG. 5 illustrates that covalently bound pMHC ligand is more stimulatory than a standard agonist pMHC on planar lipid bilayers: (a) 5C.C7 T cell photocrosslinked with BioMCC(ASA)-I-Ek, labeled with monoSAv-A647, and then added to a planar lipid bilayer presenting ICAM-1 and B7-1; (b) 5C.C7 T cell and pMHC ligands MCC(Cy5)-I-Ek added to a planar lipid bilayer presenting ICAM-1, B7-1; (c) average number of synaptic pMHC molecules from (a) and (b); (d) plots of calcium signals from each sample type (number of synaptic pMHC molecules is indicated); and (e) plots IL-2 produced by activated T cells versus number of synaptic pMHCs.

FIG. 6 shows that pMHC-engaged TCRs rapidly migrate to the center of the immunological synapse upon activation, while unengaged TCRs do not: (a) 5C.C7 T cell blasts photocrosslinked with BioMCC(ASA)-I-Ek, and labeled with monoSAv-A555 (for pMHCs) and H57-scFv-A647 (for all TCRs), and cells added to a planar lipid bilayer presenting ICAM-1 and B7-1; and (b) photocrosslinked T cells labeled with monoSAv-A555 (for pMHCs) and KJ25-Fab-A647 (for unengaged TCRs).

FIG. 7 illustrates an efficient, acid-induced peptide exchange method for I-Ek: (a) schematic illustration of the peptide-exchange reaction between Lys99ANP-I-Ek and Cy3_MCC; (b) FPLC chromatography of mixture of Lys99ANP-I-Ek and Cy3_MCC showing no peptide exchange occurred; (c) FPLC chromatography of mixture of Lys99ANP-I-Ek and Cy3_MCC showing exchange yield of about 30%; (d) mixture of Lys99ANP-I-Ek and Cy3_MCC without any UV irradiation showing exchange of Lys99ANP-I-Ek to Cy3_MCC-I-Ek.

FIG. 8 illustrates staining of 5C.C7 T cell blasts mixed with BioMCC(ASA)-I-Ek (0.5 μM) after various photoactivation times.

FIG. 9 illustrates that pMHC-TCR crosslinking and KJ25-TCR recognition are mutually exclusive: (a) flow cytometric analysis of 5C.C7 T cell blasts photocrosslinked with BioMCC(ASA)-I-Ek and stained by PE-SAv and PE-KJ25, respectively; and (b) bound PE-SAv and PE-KJ25 per cell.

FIG. 10 illustrates flow cytometric analysis of 5C.C7 T cell blasts staining signal from photocrosslinkable pMHC monomers (BioMCC(ASA)-I-Ek) is sustained over a longer time, compared with the pMHC tetramer (PE-conjugated MCC-I-Ek) staining.

FIG. 11 illustrates that photocrosslinkable pMHC monomer and pMHC tetramer can be used to detect T cell populations with differing affinities in a mixture. T cells from 5C.C7 TCR β-chain transgenic mice were primed with the MCC peptide, stained by H57-FITC and either a PE-conjugated MCC-I-Ek tetramer (a) or a photocrosslinkable BioMCC(ASA)-I-Ek monomer (b), and subjected to flow cytometric analysis.

FIG. 12 illustrates quantification of synaptic pMHC and TCR molecules in T cells photocrosslinked with pMHC ligands: (a) 5C.C7 T cells photocrosslinked with BioMCC(ASA)-I-Ek formed immunological synapses after landing on planar lipid bilayers presenting ICAM-1 and B7-1; and (b) numbers of synaptic pMHCs and TCRs by fluorescence single-molecule counting assays.

### DEFINITIONS

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As used herein, “suitable conditions” for carrying out a synthetic step are explicitly provided herein or may be discerned by reference to publications directed to methods used in synthetic organic chemistry. The reference books and treatise set forth above that detail the synthesis of reactants useful in the preparation of compounds of the present invention, will also provide suitable conditions for carrying out a synthetic step according to the present invention.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution. The term lower alkyl will be used herein as known in the art to refer to an alkyl, straight, branched or cyclic, of from about 1 to 6 carbons.

The compounds of the invention, or their pharmaceutically acceptable salts may contain one or more asymmetric centers and may thus give rise to enantiomers, diastereomers, geometric isomers, individual isomers and other stereoisomeric forms that may be defined, in terms of absolute stereochemistry, as (R)— or (S)— or, as (D)- or (L)- for amino acids. The present invention is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

### DETAILED DESCRIPTION OF THE EMBODIMENTS

As summarized above, methods for labeling and/or detecting a T cell according to specificity of an antigen T cell receptor (TCR) are provided. Also provided are monomeric MHC-peptide complexes and kits for crosslinking to a T cell according to specificity of an antigen T cell receptor (TCR). The methods and monomeric MHC-peptide complexes find use in a variety of applications related to the detection and purification of antigen-specific T cells, such as those T cells involved in tumors, infectious diseases and autoimmune diseases.

### Monomeric MHC-Peptide Complex

The monomeric MHC-peptide complex is prepared with major histocompatibility complex (MHC) protein subunits having a peptide bound in the antigen presentation site. The monomeric MHC-peptide complex specifically bind to a cognate TCR and, upon application of a stimulus, crosslink to the TCR to form a stable structure, thereby allowing for the labeling, identification and separation of T cells based on the specificity of the antigen receptor. Once the MHC-peptide complex is covalently crosslinked to the T cell, the interaction is maintained over a long period of time. An advantage to the use of a MHC-peptide complex that is monomeric rather than multimeric can be a reduction of undesirable TCR-internalization and T-cell activation.

Aspects of the invention include specifically binding a monomeric MHC-peptide complex to a T cell receptor (TCR) of the T cell and applying a stimulus to covalently crosslink the complex to the T cell. The crosslink is formed after photoactivation of a photocrosslinking moiety on the peptide. Application of the stimulus activates the photocrosslinking moiety such that it spontaneously reacts with a proximal or adjacent moiety of the TCR, to form a stable covalent crosslink.

The photocrosslinking moiety is located on the peptide of the MHC-peptide complex, at a site outside of the core TCR-recognition region of the peptide. This location is selected so that the photocrosslinking moiety does not disrupt specific binding of the peptide to the T cell receptor. In this way a TCR that recognizes a native or naturally existing MHC-peptide complex can be labeled by the methods of the invention, i.e. prior immunization with a peptide comprising the photocrosslinking moiety is not required.

The subject complexes may be adapted to label multiple T cells simultaneously by utilizing a mixture of peptides that include different antigens (e.g., core TCR-recognition regions); and/or a mixture of MHC proteins. In some embodiments, individual peptides or MHC proteins are labeled so as to be distinguished from other peptides or MHC complexes.

Photocrosslinking may occur between the peptide of the MHC-peptide complex and any convenient moiety present on the TCR protein. In some embodiments there may be no crosslinking between the peptide and the MHC of the monomeric MHC-peptide complex.

The subject monomeric MHC-peptide complex for crosslinking to a T cell according to specificity of an antigen T cell receptor (TCR) may be described by formula (I): α-β-P, where α is a soluble form of an α-chain of a class I or class II MHC protein; β is a soluble form of (i) a β-chain of a class II MHC protein, or (ii) β₂ microglobulin for a class I MHC protein; and P is a peptide comprising a TCR-recognition region and a photocrosslinking moiety attached to the peptide at a region outside of the TCR-recognition region; and where P is bound in the groove formed by two membrane distal domains of (i) the α-chain for a class I MHC protein or (ii) α-chain and the β-chains for a class II MHC protein.

### Peptides

The peptide of the monomeric MHC-peptide complex includes a core TCR-recognition region and a photocrosslinking moiety. The core TCR-recognition region of the peptide may include a T cell epitope or minimal antigenic determinant that provides for specific binding of the MHC-peptide complex to the TCR. Any T cell epitope of interest, or at least minimal antigenic determinant versions of the same, can be utilized in the subject complexes.

Generally the core TCR-recognition region is a linear amino acid sequence of 5 or more residues, such as 6 or more, 8 or more, 10 or more, 12 or more, 13 or more, 14 or more, 16 or more, or 18 or more residues, e.g., 5, 6, 7, 8, 9, 10, 11, 12, 13, 14, 15, 16, 17, or 18, or even more amino acid residues. Usually the core TCR-recognition region is between 5 and 30 amino acid residues, such as between 5 and 20 residues, between 6 and 18 amino acid residues, or between 8 and 17 residues, e.g., between 8 and 12 amino acid residues, or between 13 and 17 amino acid residues.

In some embodiments, the MHC-peptide complex comprises an MHC class I molecule, for example human HLA A, B or C; β₂ microglobulin, and a peptide with a core TCR-recognition region of between 6 and 12 amino acid residues, such as between 8 and 12 residues; and a photocrosslinking moiety outside of the recognition region.

In other embodiments, the MHC-peptide complex comprises an alpha chain and a beta chain of an MHC class II molecule, for example human HLA DP, DM, DQ, DR, DOA/B; and a peptide with a core TCR-recognition region of between 6 and 20 amino acid residues, such as between 10 and 18 residues or between 13 and 17 amino acid residues; and a photocrosslinking moiety outside of the recognition region.

The peptides may have a sequence derived from a wide variety of proteins. Many T cell epitopes are known in the art, and include a variety of autoantigens, tumor antigens, allergens, pathogen antigens, and the like, as known in the art. Any convenient epitopic sequences from a number of antigens may be utilized. Alternatively, the epitopic sequence may be empirically determined, by isolating and sequencing peptides bound to native MHC proteins, by synthesis of a series of peptides from the target sequence, then assaying for T cell reactivity to the different peptides, or by producing a series of binding complexes with different peptides and quantitating the T cell binding. For example, see De Groot et al. (2010) Methods in Microbiology 37:35-66, “Use of Bioinformatics to Predict MHC Ligands and T-Cell Epitopes: Application to Epitope-Driven Vaccine Design”, herein specifically incorporated by reference. Preparation of fragments, identifying sequences, and identifying the minimal sequence is amply described in U.S. Pat. No. 5,019,384, iss. May 28, 1991, and references cited therein. These peptide sequences may be utilized in the core T-cell recognition regions of the subject peptides.

The subject peptides may be prepared in a variety of ways. Conveniently, they can be synthesized using conventional techniques employing automatic synthesizers, or may be synthesized manually. Alternatively, DNA sequences can be prepared that encode the peptide of interest, which are cloned and expressed to provide the desired peptide for utilization in preparation of the photocrosslinking moiety-containing peptide. Peptide fragments may be produced by recombinant methods, for example as a fusion to a polypeptide with a tag for purification, allowing purification of the fusion protein by means of affinity reagents, followed by proteolytic cleavage, usually at an engineered site to yield the desired peptide fragment (see for example Driscoll et al. (1993) J. Mol. Bio. 232:342-350). The peptides may also be purified using any convenient techniques, including, for example, chromatography on ion exchange materials, separation by size, immunoaffinity chromatography and electrophoresis.

The photocrosslinking moiety is located at a position of the peptide that does not disrupt specific binding of MHC-peptide complex to the TCR. Any convenient location of the peptide may be selected for inclusion of the photocrosslinking moiety, usually the photocrosslinkable moiety is outside of the linear recognition sequence, and may be at the amino or carboxy terminus of the peptide. In some cases, the photocrosslinking moiety is attached to an amino acid residue of the peptide, where the residue is not an antigenic residue essential to the specific binding to the TCR, for example within the core recognition region.

In some embodiments, the photocrosslinking moiety is attached to an amino acid residue that is contained within the sequence of a T cell epitope, but where the residue is not involved in contacts with the TCR, and is not an essential antigenic residue of the motif that defines the minimum antigenic determinant.

In other embodiments, the photocrosslinking moiety is located at a position outside of the core TCR-recognition region of the peptide. By “a position outside of the core” is meant that the photocrosslinking moiety is connected to a region of the peptide that does not include or overlap with the core TCR-recognition region, but rather the photocrosslinking moiety is attached at a position that is adjacent to the core TCR-recognition region. In some instances, the photocrosslinking moiety is N-terminal or C-terminal to the core TCR-recognition region. In certain instances, the photocrosslinking moiety is connected to an amino acid residue that is at a position in the peptide sequence that is one or more residues N-terminal or C-terminal to the core TCR-recognition sequence. The photocrosslinking moiety may be attached to the subject peptide at any convenient location, including but not limited to: attached via a sidechain functional group (such as the amino group of a lysine residue or the thiol group of a cysteine residue or the carboxylic acid group of an aspartic acid or a glutamic acid residue); or attached via a terminal group (such as the α-amino group of an amino acid residue).

The photocrosslinking moiety may be incorporated into the peptide utilizing any convenient methods. Methods of interest include, but are not limited to bioconjugation methods, solid phase peptide synthesis methods, native chemical ligation methods, and the like. The photocrosslinking moiety may be connected to an amino acid via a linker, where the linker may include peptidic residues, non-peptidic groups or residues, or mixtures of the same.

The peptide may further include a detectable moiety or additional crosslinking moiety (e.g., as described herein). The detectable moiety or additional crosslinking moiety may also be located at a position outside of the core TCR-recognition region. The detectable moiety or additional crosslinking moiety may be connected to the peptide at any convenient position via an optional peptidic or non-peptidic linker.

As used herein, the term “linker” or “linkage” refers to a linking moiety that connects two groups and has a backbone of 20 atoms or less in length. A linker or linkage may be a covalent bond that connects two groups or a chain of between 1 and 20 atoms in length, for example of about 1, 2, 3, 4, 5, 6, 8, 10, 12, 14, 16, 18 or 20 carbon atoms in length, where the linker may be linear, branched, cyclic or a single atom. The linker may be peptidic (e.g., include one or more amino acid residues) or non-peptidic. In certain cases, one, two, three, four or five or more carbon atoms of a linker backbone may be optionally substituted with a sulfur, nitrogen or oxygen heteroatom. The bonds between backbone atoms may be saturated or unsaturated, usually not more than one, two, or three unsaturated bonds will be present in a linker backbone. The linker may include one or more substituent groups, for example with an alkyl, aryl or alkenyl group. A linker may include, without limitations, oligo(ethylene glycol); ethers, thioethers, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

As used herein, the term “cleavable linker” refers to a linker that can be selectively cleaved to produce two products. Application of suitable cleavage conditions to a molecule containing a cleavable linker that is cleaved by the cleavage conditions will produce two fragmentation products. A cleavable linker of the present invention is stable, e.g. to physiological conditions, until it is contacted with a cleavage-inducing stimulus, e.g., an agent such as an enzyme or other cleavage-inducing agent or light.

For clarity, the number of atoms that connect two groups is calculated by counting the minimum number of covalently linked atoms between the two groups, excluding atoms of the two groups themselves. When the linkage between two groups includes a cyclic moiety, the shortest path around the ring of the cyclic moiety is counted so that a minimum possible number of atoms that connect the two groups is calculated.

The subject peptide of the MHC-peptide complex may be described by formula (II):

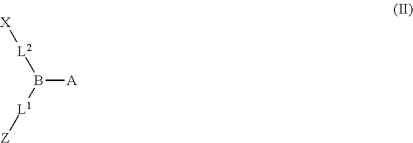

where A is the core TCR-recognition region; X is a photocrosslinking moiety; L¹ and L² are optional linkers (e.g., non-peptidic linker); B is a peptidic linker comprising one or more amino acid residues; and Z is an optional detectable moiety or an optional crosslinking moiety.

Any convenient amino acid residues may be utilized in peptidic linkers of the subject peptide. The residues may be selected to provide a linker with a desired flexibility, solubility, and number of sidechain functional groups suitable for conjugation to X and/or Z, either directly or via optional linkers. In some instances, B includes one or more amino acid residues, independently selected from G, C, S, K, D, E and beta-alanine. In some cases, the peptidic linker B further includes one or more non-peptidic groups, such as a polyethylene glycol group or a C2-C6 alkyl linker such as 6-amino-hexanoic acid.

Any convenient crosslinking moieties may be utilized in the subject peptides. A variety of protein crosslinking technologies and moieties are known to those of skill in the art and can be utilized in the subject peptides, see e.g., groups as described in G. T. Hermanson, “Bioconjugate Techniques” Academic Press, 2nd Ed., 2008. Functional groups that may be used as crosslinking moieties include, but are not limited to, active esters, isocyanates, imidoesters, hydrazides, amino groups, aldehydes, ketones, photoreactive groups, maleimide groups, alpha-halo-acetyl groups, epoxides, azirdines, and the like. In some cases, in formula (II), the optional crosslinking moiety is selected from an amino-reactive group, a sulfhydryl reactive group, a hydroxyl reactive group, an imidazolyl reactive group and a guanidinyl reactive group. In particular embodiments, the optional crosslinking moiety is selected from N-hydroxysuccinimidyl ester, sulfo-N-hydroxysuccinimidyl ester, a halo-substituted phenol ester, pentafluorophenol ester, a nitro-substituted phenol ester, an anhydride, isocyanate, isothiocyanate, an imidoester, maleimide, iodoacetyl, hydrazide, an aldehyde, an epoxide, an amino and a photoreactive linking group, A variety of reagents for crosslinking proteins may be adapted for use in the subject peptides, including but not limited to, azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-.gamma.-maleimidobutyryloxysuccinimide ester, N-hydroxy sulfosuccinimidyl-4-azidobenzoate, N-succinimidyl [4-azidophenyl]-1,3′-dithiopropionate,N-succinimidyl [4-iodoacetyl]aminobenzoate, glutaraldehyde, formaldehyde and succinimidyl 4-[N-maleimidomethyl]cyclohexane-1-carboxylate.

In some embodiments, the peptide of the MHC-peptide complex is described by formula (III):

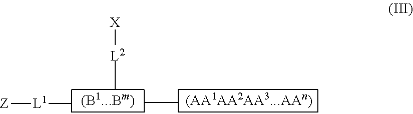

where X, Z, L¹ and L² are as described for Formula (I); (AA¹ AA² AA³ . . . AAⁿ) is a sequence of n amino acid residues from AA¹ to AAⁿ that comprises the core TCR-recognition element, wherein n is an integer from 5 to 30; (B¹ . . . B^{m}) is a sequence of m residues from B¹ to B^{m}, wherein m is an integer from 1 to 20. In certain embodiments, residue B^{m} is N-terminal to the core TCR-recognition element. In other embodiments, (B¹ . . . B^{m}) is located C-terminal to the core TCR-recognition element, such that in formula II, the peptide would be depicted as a sequence in the C→N direction. In certain embodiments, X is attached to the peptide via the B^{m}, B^{(m-1)} or B^{(m-2)} residue sidechain. In certain embodiments, X is separated from the core TCR-recognition region by one or more residues, such as by 1, 2, 3 or more residues.

The (AA¹ AA² AA³ . . . AAⁿ) region includes the core TCR-recognition region of the peptide. In some instances, (AA¹ AA² AA³ . . . AAⁿ) includes a T cell epitope (e.g., as described herein), a minimum antigenic determinant, or an analog thereof. In some embodiments, the core TCR-recognition region consists of only naturally occurring amino acids. In certain embodiments, the core TCR-recognition region consists of only underivatized amino acids, e.g., amino acids whose sidechain groups are not further conjugated to a moiety such as a crosslinkng moiety or a detectable moiety.

In some instances, n is 5 or greater, such as 8 or greater, 13 or greater, 18 or greater, such as 5, 6, 7, 8, 9, 10, 11, 12, 13, 14, 15, 16, 17, or 18, or even greater. In certain embodiments, n is an integer that ranges between 5 and 30, such as between 5 and 20, between 6 and 18, or between 8 and 17, e.g., between 8 and 12, or between 13 and 17. In some instances, in formula (II), n ranges between 8 and 12. In other instances, n ranges between 13 and 17 amino acid residues.

Any convenient amino acid residues may be utilized in the peptidic linker (B¹ . . . B^{m}). In some instances, the peptidic linker includes one or more amino acid residues, independently selected from G, C, S, K, D, E and beta-alanine. In some cases, where m is 1, (B¹ . . . B^{m}) is a single residue B¹. In other cases, m is an integer from 2 to 20, such as 2, 3, 4, 5, 6, 7, 8, 9, 10, 11, 12, 13, 14, 15, 16, 17, or 18. In certain embodiments, m ranges between 2 and 12, such as between 2 and 10, between 2 and 8, or between 2 and 6. In some instances, in formula (III), m ranges between 3 and 6.

### Photocrosslinking Moieties

Photocrosslinking moieties suitable for use in the subject peptides include any group that upon application of a light stimulus may be photoactivated for crosslinking to an adjacent compatible functional group. Any convenient photocrosslinking moieties may be utilized in the subject peptides. In some cases, the photocrosslinking moiety includes a photoreactive aryl azide group, such as described in the photoaffinity reagents and groups summarized by G. T. Hermanson, “Bioconjugate Techniques” Academic Press, 2nd Ed., 2008. Photocrosslinking moieties of interest include but are not limited to: 4-azidosalicylic acid (ASA), p-azidobenzoyl, 5-azido-2-nitrobenzoyl, 4-azido-2-nitrobenzoyl, p-azido-L-phenylalanine, m-azido-o-nitrobenzamido group, 4-azido-2-hydroxy-phenyl acid, p-azido-phenyl, 5-azido-2-nitrophenyl, 4-azido-2-nitrophenyl, p-azidophenyl, m-azido-o-nitro-phenyl, 7-azido-4-methylcoumarin, diazopyruvate, 2-diazo-3,3,3-trifluoropropionate, benzophenone, diazirine, and derivatives thereof. A variety of derivatives of the above photocrosslinking groups, such as, acyl, amido, alkyl, alkyloxy, and acyloxy derivatives or iodinated derivatives, may be utilized in adapting these groups for use in the subject peptides.

### Detectable Moieties

As used herein, the term “detectable moiety” refers a moiety that can be detected by a variety of methods including fluorescence, electrical conductivity, radioactivity, size, and the like. The detectable moiety may be of a chemical (e.g., carbohydrate, lipid, etc.), peptide or nucleic acid nature although it is not so limited. The detectable moiety may be directly or indirectly detectable. The detectable moiety can be detected directly for example by its ability to emit and/or absorb light of a particular wavelength. A detectable moiety can be detected indirectly by its ability to bind, recruit and, in some cases, cleave (or be cleaved by) another compound, thereby emitting or absorbing energy. An example of indirect detection is the use of an enzyme label that cleaves a substrate into visible products.

Any convenient detectable moiety may be utilized in the subject peptides. Detectable moieties of interest include, but are not limited to, fluorophores, dyes, enzymes or enzyme substrates, chemiluminescers, specific binding moieties or their partners, particles, radioisotopes, affinity tags or other directly or indirectly detectable agent. In certain cases, the detectable moiety has a light detectable characteristic, e.g., a fluorophore, such as fluorescein isothiocyanate (FITC), Texas Red, Cy3, Cy5, phycoerythrin, allophycocyanin, 5,6-carboxymethyl fluorescein, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7.

The detectable moieties may also be specific binding moieties such as receptors, antibodies or antibody fragments or their corresponding antigen, epitope or hapten, or ligand binding partners. Detection of such moieties is accomplished by any convenient techniques. In certain embodiments, the detectable moiety is biotin. In some instances, the detectable moiety is an affinity tag that is suitable for use in methods of purification of T cells, e.g., by binding to a chromatography column. The detectable moiety may be utilized in the separation and/or purification of labeled T cell from unlabeled T cells.

### MHC Molecule

The complex is prepared with major histocompatibility complex protein subunits having a homogeneous population of peptides bound in the antigen presentation site.

The binding complex has the formula (α-β-P), where α is an alpha-chain of a class I or class II MHC protein β is a beta chain, herein defined as the beta chain of a class II MHC protein or β₂-microglobulin for a MHC class I protein. The p-MHC complex specifically binds to a T cell receptor having the cognate antigenic specificity.

The MHC proteins may be from any mammalian or avian species, e.g. primate sp., particularly humans; rodents, including mice, rats and hamsters; rabbits; equines, bovines, canines, felines; etc. Of particular interest are the human HLA proteins, and the murine H-2 proteins. Included in the HLA proteins are the class II subunits HLA-DPα, HLA-DPβ, HLA-DQα, HLA-DQβ, HLA-DRα and HLA-DRβ, and the class I proteins HLA-A, HLA-B, HLA-C, and β₂-microglobulin. Included in the murine H-2 subunits are the class I H-2K, H-2D, H-2L, and the class II I-Aα, I-Aβ, I-Eα and I-Eβ, and β₂-microglobulin. A variety of MHC proteins may be utilized in the subject compositions and methods.

In some embodiments, the MHC protein subunits are a soluble form of the normally membrane-bound protein. The soluble form is derived from the native form by deletion of the transmembrane domain. Conveniently, the protein is truncated, removing both the cytoplasmic and transmembrane domains. The protein may be truncated by proteolytic cleavage, or by expressing a genetically engineered truncated form.

For class I proteins, the soluble form may include the α1, α2 and α3 domains. In some cases, ten or less, such as five or less, or none of the amino acids of the transmembrane domain are included. The deletion may extend as much as about 10 amino acids into the α3 domain. In some instances, none of the amino acids of the α3 domain are deleted. The deletion may be such that it does not interfere with the ability of the α3 domain to fold into a disulfide bonded structure. The class I β-chain, β₂-microglobulin, lacks a transmembrane domain in its native form, and need not be truncated. In some instances, no Class II subunits are used in conjunction with Class I subunits.

Soluble class II subunits may include the α1 and α2 domains for the α-subunit, and the β1 and β2 domains for the β subunit. In some cases, ten or less, such as five or less, or none of the amino acids of the transmembrane domain are included. The deletion may extend as much as about 10 amino acids into the α2 or β2 domain, preferably none of the amino acids of the α2 or β2 domain will be deleted. The deletion will be such that it does not interfere with the ability of the α2 or β2 domain to fold into a disulfide bonded structure.

In some instances, a small number of amino acids are introduced at the polypeptide termini, e.g., not more than 20, more usually not more than 15. The deletion or insertion of amino acids may be as a result of the needs of the construction, providing for convenient restriction sites, addition of processing signals, ease of manipulation, improvement in levels of expression, or the like. In addition, one or more amino acids may be substituted with a different amino acid for similar reasons, e.g., not substituting more than about five amino acids in any one domain.

The alpha and beta subunits may be separately produced and allowed to associate in vitro to form a stable heteroduplex complex (see e.g., Altman et al. (1993, PNAS. 90: 10330-10334) or Garboczi et al. (1992) PNAS. 89:3429-3433) or both of the subunits may be expressed in a single cell. An exchangeable peptide may be originally included on the complex, which is then exchanged with the peptide comprising the photocrosslinking moiety. An alternative strategy is to engineer a single molecule having both the alpha and beta subunits. A “single-chain heterodimer” is created by fusing together the two subunits using a short peptide linker, e.g. a 15 to 25 amino acid peptide or linker. See Bedzyk et al. (1990) J. Biol. Chem. 265:18615 for similar structures with antibody heterodimers. The soluble heterodimer may also be produced by isolation of a native heterodimer and cleavage with a protease, e.g. papain, to produce a soluble product.

In one embodiment, soluble subunits are independently expressed from a DNA construct encoding a truncated protein. For expression, the DNA sequences are inserted into an appropriate expression vector, where the native transcriptional initiation region may be employed or an exogenous transcriptional initiation region, i.e. a promoter other than the promoter which is associated with the gene in the normally occurring chromosome. The promoter may be introduced by recombinant methods in vitro, or as the result of homologous integration of the sequence into a chromosome. A wide variety of transcriptional initiation regions are known for a wide variety of expression hosts, where the expression hosts may involve prokaryotes or eukaryotes, particularly E. coli, B. subtilis, mammalian cells, such as CHO cells, COS cells, monkey kidney cells, lymphoid cells, particularly human cell lines, and the like. Generally a selectable marker operative in the expression host will be present.

Of particular interest are expression cassettes comprising a transcription initiation region, the gene encoding the subject MHC subunit, and a transcriptional termination region, optionally having a signal for attachment of a poly A sequence. Suitable restriction sites may be engineered into the termini of the MHC subunit, such that different subunits may be put into the cassette for expression. Restriction sites may be engineered by various means, e.g. introduction during polymerase chain reaction, site directed mutagenesis, etc.

The subunits are expressed in a suitable host cell, and, if necessary, solubilized. The two subunits are combined with an antigenic peptide and allowed to fold in vitro to form a stable heterodimer complex with intrachain disulfide bonded domains. The peptide may be included in the initial folding reaction, or may be added to the empty heterodimer in a later step. Usually the MHC binding site will be free of peptides prior to addition of the target antigenic peptide.

The MHC heterodimer may bind the peptide in the groove formed by the two membrane distal domains, either α2 and al for class I, or α1 and β1 for class II. The bound peptide may be substantially homogenous, that is, there will be less than about 10% of peptide impurities, such as less than about 5%, or less than about 1%.

Any convenient conditions that permit folding and association of the subunits and peptide may be utilized, see for example Altman et al. (1993) and Garboczi et al. (1992). As one example of permissive conditions, roughly equimolar amounts of solubilized alpha and beta subunits are mixed in a solution of urea. Refolding is initiated by dilution or dialysis into a buffered solution without urea. Peptides are loaded into empty class II heterodimers at about pH 5 to 5.5 for about 1 to 3 days, followed by neutralization, concentration and buffer exchange.

### Methods

As summarized above, aspects of the invention include methods for labeling and/or detecting a T cell according to specificity of an antigen T cell receptor (TCR). The subject methods include specifically binding a MHC-peptide complex (e.g., as described above) to a TCR and applying a stimulus to crosslink the MHC-peptide complex to the TCR.

As such, aspects of the method include contacting a TCR, generally when present on the surface of a T cell, with a monomeric MHC-peptide complex (e.g., as described above) under conditions by which the monomeric MHC-peptide complex specifically binds the TCR. The core TCR-recognition region of the peptide in the complex provides for binding specificity. Another aspect of the method includes applying a stimulus to crosslink the MHC-peptide complex to the T cell. Generally the peptide is not covalently crosslinked to the MHC in the MHC-peptide complex.

The MHC-peptide complex may be free in solution, or may be attached to an insoluble support. Examples of suitable insoluble supports include beads, e.g. magnetic beads, membranes and microtiter plates. In some cases, these may be made of glass, plastic (e.g. polystyrene), polysaccharides, nylon or nitrocellulose. In some cases, the MHC-peptide complex is labeled with a detectable moiety, so as to be directly detectable, or is used in conjunction with secondary labeled reagents which specifically binds the complex.

Any convenient protocol for contacting the T cell with the monomeric MHC-peptide complex in a sample may be employed. The particular protocol that is employed may vary, e.g., depending on whether the sample is in vitro or in vivo.

Samples for use in the methods of the invention may be obtained from a variety of sources, particularly blood, although in some instances samples such as bone marrow, lymph, cerebrospinal fluid, synovial fluid, and the like may be used, or cultured T cells. Such samples can be separated by centrifugation, elutriation, density gradient separation, apheresis, affinity selection, panning, FACS, centrifugation with Hypaque, etc. prior to analysis, and often a mononuclear fraction (PBMC) will be used. Once a sample is obtained, it can be used directly, frozen, or maintained in appropriate culture medium for short periods of time.

In some cases, the assay will measure the binding in a patient sample, usually blood derived, generally in the form of plasma or serum and the subject monomeric MHC-peptide complex. The patient sample may be used directly, or diluted as appropriate, usually about 1:10 and usually not more than about 1:10,000. Assays may be performed in any physiological buffer, e.g. PBS, normal saline, HBSS, dPBS, etc.

Various media can be employed to maintain cells. The samples may be obtained by any convenient procedure, such as the drawing of blood, venipuncture, biopsy, or the like. Usually a sample will comprise at least about 10² cells, at least about 10³ cells, at least about 10⁴, 10⁵ or more cells. Often the samples will be from human patients, although animal models may find use, e.g. equine, bovine, porcine, canine, feline, rodent, e.g. mice, rats, hamster, primate, etc. Generally from about 0.001 to 1 ml of sample, diluted or otherwise, is sufficient, usually about 0.01 ml sufficing. The incubation time should be sufficient for T cells to bind the subject complex. Generally,.

[symbol]

The labeling reagent of the invention is added to a suspension of cells, and incubated for a period of time sufficient to bind the available TCR. The incubation will usually be from about 0.1 to 3 hr, usually 1 hr sufficing. It is desirable to have a sufficient concentration of labeling reagent in the reaction mixture, such that the efficiency of the separation is not limited by lack of reagent. The appropriate concentration can be determined by titration. Various media find use in the labeling. If viable cells are desired, e.g. after a separation procedure, the medium will maintain the viability of the cells, e.g. phosphate buffered saline containing from 0.1 to 0.5% BSA. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, etc.

Where a second stage labeling reagent is used, the cell suspension may be washed and resuspended in medium as described above prior to incubation with the second stage reagent. Alternatively, the second stage reagent may be added directly into the reaction mix.

The labeled cells can be quantitated as to the expression of a TCR of interest. It is particularly convenient in a clinical setting to perform the immunoassay in a self-contained apparatus. Alternatively various microscopic, flow cytometry, etc. methods find use. Techniques providing accurate enumeration include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Flow cytometry, or FACS, can also be used to separate cell populations based on the intensity of fluorescence, as well as other parameters.

Flow cytometry may also be used for the separation of a labeled subset of T cells from a complex mixture of cells. The cells may be collected in any appropriate medium which maintains the viability of the cells, usually having a cushion of serum at the bottom of the collection tube. Various media are commercially available as described above. The cells may then be used as appropriate.

Alternative means of separation utilize the subject complex bound directly or indirectly to an insoluble support, e.g. column, microtiter plate, magnetic beads, etc. The cell sample is added to the binding complex. The complex may be bound to the support by any convenient means. After incubation, the insoluble support is washed to remove non-bound components. From one to six washes may be employed, with sufficient volume to thoroughly wash non-specifically bound cells present in the sample. In particular the use of magnetic particles to separate cell subsets from complex mixtures is described in Miltenyi et al. (1990) Cytometry 11:231-238.

The insoluble supports may be any compositions to which the multimeric binding complex can be bound, which is readily separated from soluble material, and which is otherwise compatible with the overall method of measuring T cells.

Generally the number of bound T cells detected will be compared to control samples from samples having a different MHC context or antigen specificity, e.g. T cells from an animal that does not express the MHC molecule used to make the binding complex.

### Stimulus

Aspects of the methods include applying a suitable stimulus to the sample after contacting with the labeling reagent. Any suitable stimulus may be utilized to crosslink the monomeric MHC-peptide complex of the invention to a T cell. In some cases, the stimulus is light, e.g., a photon. Any suitable source of light may be used in the subject methods for application of the stimulus. Light sources suitable for use in the subject methods include, but are not limited to, UV lamps (e.g., a xenon flash lamp) and laser light sources (e.g., ultraviolet lasers) that irradiate light at an appropriate wavelength suitable for absorption by the probe. Laser light sources include the frequency-doubled ruby laser, which produces a, e.g., 200 mJ pulse at 347 nm in 50 ns, and a nitrogen laser (producing e.g., 200 mJ at 337 nm), where sufficient intensity can be achieved by focusing the light through a microscope objective. Any suitable lasers may be configured to produce brief (ns) pulses of monochromatic light of intensity sufficient to modify a probe in a sample. Xenon flash lamps produce a broad spectrum, from 250 to 1500 nm, and may produce pulses of about 1 ms. Filters may be placed in the light path to narrow the spectrum and remove wavelengths (e.g., <300 nm). In certain cases, after filtering, the total output of the lamp may between about 300 and about 400 nm (e.g., between about 320 nm to about 380 nm, between about 330 nm to about 370 nm, or between about 340 nm to about 360 nm) can be configured to produce between about 50 mJ and about 250 mJ (e.g., about 200 mJ) light of intensity sufficient to modify a probe in a sample.

The light source may have a spectral energy distribution suitable for the particular photocrosslinking moiety being used in conjunction with the complex. In some cases, photocrosslinking is dependent on the wavelength of the irradiating light, its intensity and duration. A bulb providing a light intensity at the sample in the range of about 0.2 to about 10 mW/cm² at 365 nm with a 10 nm bandpass may be suitable for such purposes. Light sources of interest include, but are not limited to: chemists' mercury spot lamps with 110 watts BL9 phosphorescent bulbs, 100 W xenon arc lamp which is passed through Hoya 340 and Schott WG 305 filters before illuminating the sample, one or more flashes (e.g., a 50-ns flash) from a frequency-doubled ruby laser that delivers 347 nm light with an average energy of 90 mJ (range 83-104 mJ). It should be understood that the aforementioned wavelength range may be selected as a compromise between using shorter wavelengths that may damage components of the sample (e.g., wavelengths below 300 nm) and using longer wavelengths that may be less effective at photocrosslinking (e.g., wavelengths above 500 nm). Light having other spectral energy distributions may be required for activating other photocrosslinking moieties. Such other energy distributions are readily available, or can be readily determined using any convenient method.

A variety of methods for supplying uniform illumination, controlling illumination intensity, controlling illumination time, controlling sample temperature, and spatiotemporal control of illumination may be used. As used herein, the terms illumination and irradiation are used interchangeably. In some embodiments, the illumination time is about 30 sec or more, such as about 1 minute or more, about 2 minutes or more, about 3 minutes or more, about 5 minutes or more, about 10 minutes or more, about 20 minutes or more, about 30 minutes or more, about 60 minutes or more, or even more. In certain embodiments, the illumination time includes flash photolysis pulses from a laser of nanosecond, picosecond or femtosecond pulse width. The light source may be directed onto the sample using any convenient method. In some cases, the light source is directed via the optical path of a microscope, where the light can be controlled spatially (e.g., by focusing the light into a small spot at a particular location).

T Cells

The subject monomeric MHC-peptide complexes (labeling reagent) and methods are used to label, detect and/or separate antigen specific T cells. The T cells may be from any source, usually having the same species of origin as the MHC molecules. The T cells may be from an in vitro culture, or a physiologic sample. In many cases, the physiologic samples employed will be blood or lymph, but samples may also involve other sources of T cells, particularly where T cells may be invasive. Thus, other sites of interest are tissues, or associated fluids, as in the brain, lymph node, neoplasms, spleen, liver, kidney, pancreas, tonsil, thymus, joints, synovia, and the like. The sample may be used as obtained or may be subject to modification, as in the case of dilution, concentration, or the like. Prior treatments may involve removal of cells by various techniques, including centrifugation, using Ficoll-Hypaque, panning, affinity separation, using antibodies specific for one or more markers present as surface membrane proteins on the surface of cells, or any other technique that provides enrichment of the set or subset of cells of interest.

TCR specificity determines what antigens will activate that particular T cell. In general terms, T helper cells express CD4 on their surface, and are activated by binding to a complex of antigenic peptide and Class II MHC molecule. Cytolytic T cells may express CD8 on their surface, and are activated by binding to a complex of antigenic peptide and Class I MHC molecule. The specificity of the T cell antigen receptor is the combination of peptide and MHC molecule that binds to that particular TCR with sufficient affinity to activate the T cell. A variety of MHC-peptide complexes may be utilized in the binding complex. Complexes of class I MHC molecules may be used to detect CD8⁺ T cells, and class II complexes may be used to detect CD4⁺ T cells. Quantitation of T cells may be performed to monitor the progression of a number of conditions associated with T cell activation, including autoimmune diseases, graft rejection, vital infection, bacterial and protozoan infection. T cells having a particular antigenic specificity may be separated from complex mixtures, particularly biological samples, utilizing the subject methods. In this way selective depletion or enrichment of particular T cells can be made.

### Utility

The monomeric MHC-peptide complexes, peptides and methods of the invention, e.g., as described above, find use in a variety of applications. Applications of interest include, but are not limited to: research applications, diagnostic applications and therapeutic applications. Methods of the invention find use in a variety of different applications including any convenient application related to the detection and/or purification of T cells. In such cases, the subject MHC-peptide complexes and methods may be used to label, detect and/or separate a T-cell of interest in a sample.

The subject monomeric MHC-peptide complexes and methods find use in a variety of diagnostic applications, including but not limited to, the diagnosis of a disease condition associated with the T-cell, e.g., in vitro diagnostics or in vivo diagnostics. Such applications are useful in diagnosing or confirming diagnosis of a disease condition, or susceptibility thereto, determining the proper course of treatment for a patient suffering from a disease condition. The methods are also useful for monitoring disease progression and/or response to treatment in patients who have been previously diagnosed with the disease. Diagnostic applications of interest include diagnosis of disease conditions, including but not limited to: tumors, infectious diseases and autoimmune diseases.

Detection of T cells is of interest in connection with a variety of conditions associated with T cell activation. Such conditions include autoimmune diseases, e.g. multiple sclerosis, myasthenia gravis, rheumatoid arthritis, type 1 diabetes, graft vs. host disease, Grave's disease, etc.; various forms of cancer, e.g. carcinomas, melanomas, sarcomas, lymphomas and leukemias. Various infectious diseases such as those caused by viruses, e.g. HIV-1, hepatitis, herpesviruses, enteric viruses, respiratory viruses, rhabdovirus, rubeola, poxvirus, paramyxovirus, morbillivirus, etc. are of interest. Infectious agents of interest also include bacteria, such as Pneumococcus, Staphylococcus, Bacillus. Streptococcus, Meningococcus, Gonococcus, Eschericia, Klebsiella, Proteus, Pseudomonas, Salmonella, Shigella, Hemophilus, Yersinia, Listeria, Corynebacterium, Vibrio, Clostridia, Chlamydia, Mycobacterium, Helicobacter and Treponema; protozoan pathogens, and the like. T cell associated allergic responses may also be monitored, e.g. delayed type hypersensitivity or contact hypersensitivity involving T cells.

Also of interest are conditions having an association with a specific peptide or MHC haplotype, where the subject complexes may be used to track the T cell response with respect to the haplotype and antigen. A large number of associations have been made in disease states that suggest that specific MHC haplotypes, or specific protein antigens are responsible for disease states. In such cases, direct detection of reactive T cells in patient samples is of interest. Detection and quantitation with the subject complexes allows such direct detection. As examples, the activity of cytolytic T cells against HIV infected CD4+ T cells may be determined using the subject methods. The association of diabetes with the DQB1*0302 (DQ3.2) allele may be investigated by the detection and quantitation of T cells that recognize this MHC protein in combination with various peptides of interest. The presence of T cells specific for peptides of myelin basic protein in conjunction with MHC proteins of multiple sclerosis patients may be determined. The antigenic specificity may be determined for the large number of activated T cells that are found in the synovial fluid of rheumatoid arthritis patients. It will be appreciated that the subject methods are applicable to a number of diseases and immune-associated conditions.

The isolation of antigen specific T cells finds a wide variety of applications, including therapeutic applications. The isolated T cells may find use in the treatment of cancer as in the case of tumor-infiltrating lymphocytes. Specific T cells may be isolated from a patient, expanded in culture by cytokines, antigen stimulation, etc., and replaced in the autologous host, so as to provide increased immunity against the target antigen. A patient sample may be depleted of T cells reactive with a specific antigen, to lessen an autoimmune response.

The DNA sequence of single T cell receptors having a given antigen specificity is determined by isolating single cells by the subject separation method. Conveniently, flow cytometry may be used to isolate single T cell, in conjunction with single cell PCR amplification. In order to amplify unknown TCR sequences, various amplification protocols may be used.

### Kits

Aspects of the invention further include kits, where the kits include one or more components employed in methods of the invention, e.g., peptides, MHC molecules, MHC-peptide complexes, T cells, and a stimulus-applying component. In some embodiments, the subject kit includes a monomeric MHC-peptide complex (e.g., as described herein), or precursor components thereof, and one or more components selected from a light source, a T cell, a buffer, instructions.

A variety of components suitable for use in specifically binding and photocrosslinking a MHC-peptide complex to a T cell may find use in the subject kits. Any of the components described herein may be provided in the kits. The subject kits may further comprise additional reagents which are required for or convenient and/or desirable to include in the reaction mixture prepared during the subject methods, where such reagents include buffers for specifically binding complexes to T cells; reagents, buffers for use in photocrosslinking complexes to T cells; and the like.

Kits may also include tubes, buffers, etc., and instructions for use. The various reagent components of the kits may be present in separate containers, or some or all of them may be pre-combined into a reagent mixture in a single container, as desired.

The stimulus-applying component may be any suitable component (e.g., equipment) that finds use in the application of a stimulus to a sample (e.g., irradiation of light). In certain cases, the stimulus-applying component is a UV light source. For example, components suitable for use in application of stimuli to a sample, e.g., a light source providing spatiotemporal control of irradiation.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another form of these instructions is a computer readable medium, e.g., diskette, compact disk (CD), etc., on which the information has been recorded. Yet another form of these instructions that may be present is a website address which may be used via the internet to access the information at a removed site.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric

### General Methods and Materials

The following protocols can be adapted for use with any suitable probe.

Mice and Cell Culture.

5C.C7 TCR β chain and TCR αβ chain transgenic mice and 2B4 TCR αβ transgenic mice, all on the B10.BR background, are bred and maintained. Lymphocytes from these mice are stimulated with 10 μM MCC peptides (ANERADLIAYLKQATK) in RPMI 1640 medium containing 10% FCS. Cells are split on the second day of culture in RPMI 1640 medium containing 50 units/mL of recombinant mouse IL-2 (R&D Systems), and thereafter are split as necessary for 5 days. T cell blasts are used for staining and/or activation assays 6-8 days after extraction. The AND TCR transgenic primary T cells are primed with irradiated (1500 rads) B10.BR splenocytes and MCC peptides (10 μM) in RPMI 1640 medium containing 10% FCS, and are cultured and used for staining. The OT-1/Rag1 KO mouse is obtained from Taconic Farms (line 4175-F). Naïve T cells isolated from OT-1/Rag1 KO mice are used directly for staining.

Peptide synthesis, modification, and purification. The exchangeable peptides Lys99ANP (ANERADLIAYL[ANP]QATK) and OVA263ANP (SIINFE[ANP]L) are synthesized. The Fmoc-protected ANP residue is obtained from Advanced ChemTech (Louisville, Ky.). Crude cysteine-containing peptides, including BioMCC(Cys) (Biotin-Ahx-SGGGSGGGSCGGIAYLKQATK, underlined is the MHC and TCR recognition region), Bio102S(Cys) (Biotin-Ahx-SGGGSGGGSCGGIAYLKQASK), BioER60(Cys) (Biotin-Ahx-SGGGSGGGSCGGIYFSPANKKL), OVA(Cys) (SIINFEKLGGGGSGCS) and SIAAFASL(Cys) (SIAAFASLGGGGSGCS) are synthesized. Peptides are purified using a Rainin Dynamax HPLC system and a reverse-phase C18 column. Purified peptides are lyophilized, dissolved in dimethylformamide (DMF), and reacted with S-[2-(4-Azidosalicylamido)ethylthio]-2-thiopyridine (AET; Toronto Research Chemicals) in a 50 mM sodium phosphate buffer (pH=7) to introduce 4-azidosalicyclic acid (ASA) onto the side chain of cysteine residues. The modified peptides are purified by reverse-phase HPLC, lyophilized, dissolved in DMSO, and stored in the dark at −20° C.

Protein Expression, Labeling, and Purification.

ICAM-1, B7-1, H57 scFv, MCC(Cy5)-I-Ek are produced as described by Huppa, J. B., et al. (TCR-peptide-MHC interactions in situ show accelerated kinetics and increased affinity. Nature 463, 963-967 (2010)). KJ25 Fab′ is produced from antibody KJ25 by papain digestion using a Fab preparation kit. Purified KJ25 Fab′ was labeled by Alexa-647 using Alexa-647 succinimidyl esters. The construct for monoSAv production is provided. A TEV protease recognition sequence is inserted between the gene encoding an alive streptavidin subunit and the gene encoding His6-tag. MonoSAv with a His6-tagged tail is produced by an in vitro refolding protocol as described by Howarth, M., et al. (A monovalent streptavidin with a single femtomolar biotin binding site. Nat Methods 3, 267-273 (2006)), and its His6-tag is cleaved using TEV protease. Purified monoSAv is labeled by Alexa-647 using Alexa-647 succinimidyl esters, and purified by size-exclusion chromatography. The dye/protein ratio is determined to be 1.48 based on colorimetric measurements.

Lys99ANP-I-Ek Production and Peptide Exchange Reactions.

The His6-tagged 99ANP-1-Ek is obtained by in vitro refolding from the inclusion bodies of 1-Ek α and β subunits and the peptide Lys99ANP, using a protocol as described by Rock, E. P., and Altman et al. (Formation of functional peptide complexes of class II major histocompatibility complex proteins from subunits produced in Escherichia coli. Proc Natl Acad Sci USA 90, 10330-10334 (1993)). To exchange Lys99ANP with BioMCC(ASA) or other photocrosslinkable peptides, Lys99ANP-I-Ek in PBS (10 μM) is mixed with the new peptide (100 μM), and the pH is adjusted to 5.2 using sodium citrate (50 mM). The exchanging reaction is allowed to occur for 24 hours at room temperature, and the new peptide-I-Ek is purified by size-exclusion chromatography. The whole process, including peptide exchange and protein purification, is carried out in the dark to protect the photocrosslinker.

Photocrosslinking Between T Cells and pMHCs.

5C.C7 T cell blasts (1×106) are incubated with photocrosslinkable peptide-I-Ek in 20 μL FACS buffer (PBS, 1% FCS, 2 mM EDTA, and 0.1% sodium azide) or imaging media (HBSS, 2% FCS, 0.5 mM CaCl₂, 0.1 mM MgCl₂) in a 96-well round-bottom plate on ice. After a 30-min incubation, cells are exposed to 365 nm UV light for 5 minutes (Stratalinker 2400, 5×15 watt bulbs). For FACS analysis, cells were washed by FACS buffer (200 μL, 3 times) to remove unbound pMHCs, and then subjected to the PE-SAv staining as describe herein. For live-cell imaging, cells are washed extensively by imaging media (800 μL, 6 times) to remove unbound pMHCs, and then subjected to fluorescent labeling (if necessary) and fluorescence imaging as described below.

FACS Analysis.

T cells photocrosslinked with pMHCs are incubated with PE-SAv (to label the biotinylated pMHC crosslinked to TCR) and FITC-H57 (to label the mouse TCR β chain) in FACS buffer for 30 minutes. Cells are washed with FACS buffer (200 μL, 3 times), and then subjected to flow cytometric analysis on a Cytomics FC500 (Beckman Coulter) or a LSR II (BD Biosciences). Data is analyzed with FlowJo software. Live cells are identified by forward-scatter and side-scatter profiles.

Calcium Imaging.

T cells (1×10⁶) are loaded with 1 mM Fura-2 AM dye (Invitrogen) in complete RPMI 1640 media for 30 minutes at room temperature, and washed twice using imaging media (10 mL). Cells are then photocrosslinked with pMHCs using the protocol described herein, and resuspended in 50 μL cold imaging media, and kept on ice until imaging. The imaging experiment is carried out using a Zeiss Axiovert S100TV microscope equipped with a 40× Fluar objective (numerical aperture 1.3) and a CoolSNAP HQ CCD camera (Roper Scientific). Cells (5 μL) are loaded into imaging media (300 μL) in a Labtek eight-well chambered cover slip (Nunc) on a humidified stage at 37° C. and 5% CO₂. Imaging acquisition is controlled by Metamorph (Universal Imaging Corporation). Signals from fura-2 (ex 340 nm and 380 nm, em 510 nm/80 nm) are collected at 6 second intervals for up to 20 minutes. The intracellular calcium concentration is determined from the fura-2 excitation ratio (340 nm/380 nm) using Metamorph. To multimerize pMHC-TCR pairs, SAv-QD705 (100 nM, 2 μL, Invitrogen) is added to cells in the chamber immediately before imaging. For experiments on a planar glass-supported lipid bilayer, the lipid bilayer is prepared as described by Huppa, J. B., et al. (TCR-peptide-MHC interactions in situ show accelerated kinetics and increased affinity. Nature 463, 963-967 (2010)).

Enzyme-Linked Immunosorbent Assay (ELISA) for IL-2 Production.

5C.C7 T cell blasts (1×10⁵) that are photocrosslinked with BioMCC(ASA)-I-Ek ligands are loaded onto a planar glass-supported bilayer with 300 μL imaging media. The lipid bilayer is embedded with ICAM-1 and B7-1. In parallel, plain T cells are loaded onto a planar lipid bilayer presenting ICAM-1, B7-1, and MCC(Cy5)-I-Ek. Cells are incubated for 16 hours at 37° C., and supernatants are removed. The amount of IL-2 in the supernatant is assessed by ELISA using rat anti-mouse IL-2 antibody pairs and Europium-labeled streptavidin.

Live-Cell Imaging of TCR Mobility.

5C.C7 T cell blasts are photocrosslinked to BioMCC(ASA)-I-Ek as described herein. Cells are mixed with monoSAv-A555 and H57-scFv-A647 (or KJ25-Fab-A647) on ice. After a 30 minute incubation, cells are washed with cold imaging media (800 μL×3), and then resuspended in 50 μL cold imaging media. Right before the fluorescence imaging, cells (1×105) are added to the planar lipid bilayer that is embedded with ICAM-1 and B7-1 and that is covered with imaging media (300 μL, room temperature). Cells are then illuminated in TIRF microscopy mode using a Zeiss Axiovert 200M microscope equipped with a 100× objective (numerical aperture 1.46) and using an argon ion gas (Stabilite 2017-AR) and krypton ion gas continuous-wave laser (Stabilite-KR; Spectra Physics). Emission is analyzed with a Dual-View beam splitter (630dcxr, HQ575/40m, HQ680/50m; Optical Insights), and images are acquired with a back-illuminated EMCCD Cascade II:512 camera (Roper Scientific).

### Results

Design and Synthesis of Photocrosslinkable Antigenic Peptides.

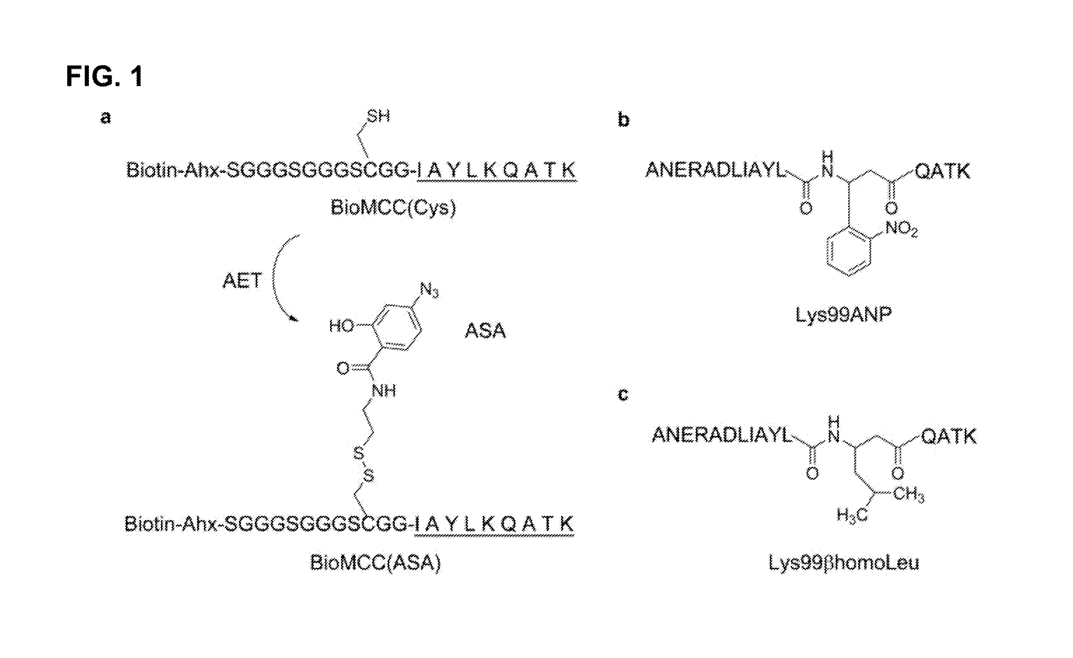

As a source of physiological CD4+ T cell blasts T cells from 5C.C7 TCR transgenic mice were used, which specifically recognize the moth cytochrome c peptide (MCC 88-103) bound to the murine MHC class II molecule I-Ek and cultured with antigen for 6-8 days. Three peptides were selected for the test, including MCC (an agonist peptide), 102S (a weak agonist), and ER60 (an endogenous peptide). They all bind strongly to I-Ek, but the formed complexes interact with the 5C.C7 TCR with varied affinities: 1) MCC-I-Ek: K_{D}=22.9 μM, t½=5.77 seconds; 2) 102S-I-Ek: K_{D}=206 μM, t_{1/2}=0.83 second; and 3) ER60-I-Ek: the K_{D} and t_{1/2} are below the detection limit of surface plasmon resonance (SPR). Next, photocrosslinkable derivatives of these peptides were generated via solid-phase peptide synthesis and chemical modification (FIG. 1a). 4-azidosalicyclic acid (ASA) was used as the photocrosslinker. To ensure that it does not interfere with the TCR recognition yet still stays close enough to capture the engaged TCR, the ASA group was introduced at a position just outside the core TCR-recognition region in the peptides. This was done by first synthesizing a peptide derivative that contains a cysteine residue at a position adjacent to the MHC-binding region, and then conjugating ASA to this cysteine (FIG. 1a). For the ease of detection, all three peptides also contain a biotin at their N-termini, and they are termed as BioMCC(ASA), Bio102S(ASA) and BioER60(ASA).

Producing Photocrosslinkable Peptide-I-Ek Complexes Using an Acid-Induced Peptide-Exchange Method.

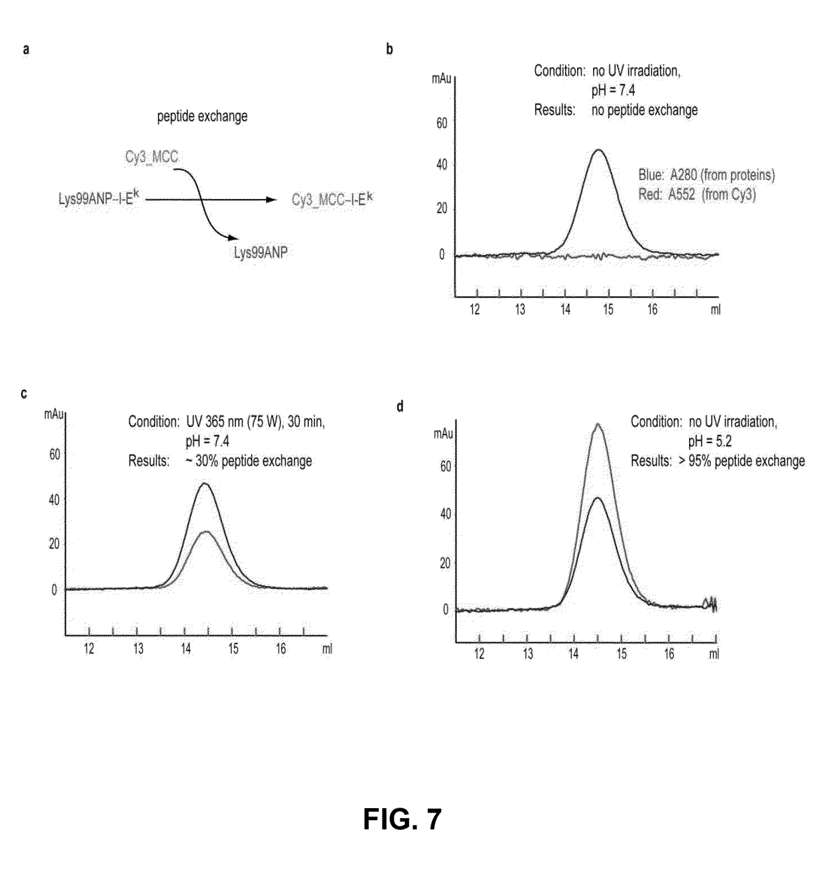

Photocrosslinkable peptide-I-Ek complexes were prepared. Since ASA is labile when exposed to light and reducing agents, conventional in vitro refolding protocols could not be used. The UV-mediated peptide-exchange method for pMHC class I molecules described by Schumacher et al. (e.g., Toebes, M., et al. Design and use of conditional MHC class I ligands. Nat Med 12, 246-251 (2006)) was adapted to develop a similar method for I-Ek. A derivative of MCC peptide (Lys99ANP, FIG. 1b) was synthesized, in which the TCR recognition residue Lys99 was replaced by a photocleavable amino acid 3-amino-3-(2-nitrophenyl)propionic acid (ANP). Lys99ANP-I-Ek was produced via in vitro refolding using Lys99ANP and inclusion bodies of 1-Ek α and β subunits. Next, the efficiency of UV-induced peptide exchange was assessed using Lys99ANP-I-Ek and a Cy3-labeled MCC peptide (Cy3_MCC). The yield of exchange was determined by FPLC purification and colorimetric measurements (FIG. 7). After a 30-minute exposure to UV light (Stratalinker 2400, 5×15 watt bulbs, 365 nm), 30% of Lys99ANP-I-Ek was converted into Cy3_MCC-I-Ek.

A variety of conditions were screened. The Lys99ANP→Cy3_MCC exchange was almost quantitative under an acidic condition (pH=5.2), even without any UV irradiation. MCC peptide derivatives containing other β-homo amino acids at the 99th position were investigated as exchangeable under acidic conditions. This was confirmed to be true on a Lys99→β-homoLeu derivative of MCC peptide (FIG. 1c). The acid-mediated peptide-exchange method described herein does not require the use of proteases. Using this method, all three photocrosslinkable pMHC ligands, including BioMCC(ASA)-I-Ek, Bio102S(ASA)-I-Ek and BioER60(ASA)-I-Ek were produced.

Detecting Antigen-Specific T Cells Using a Photocrosslinkable pMHC.

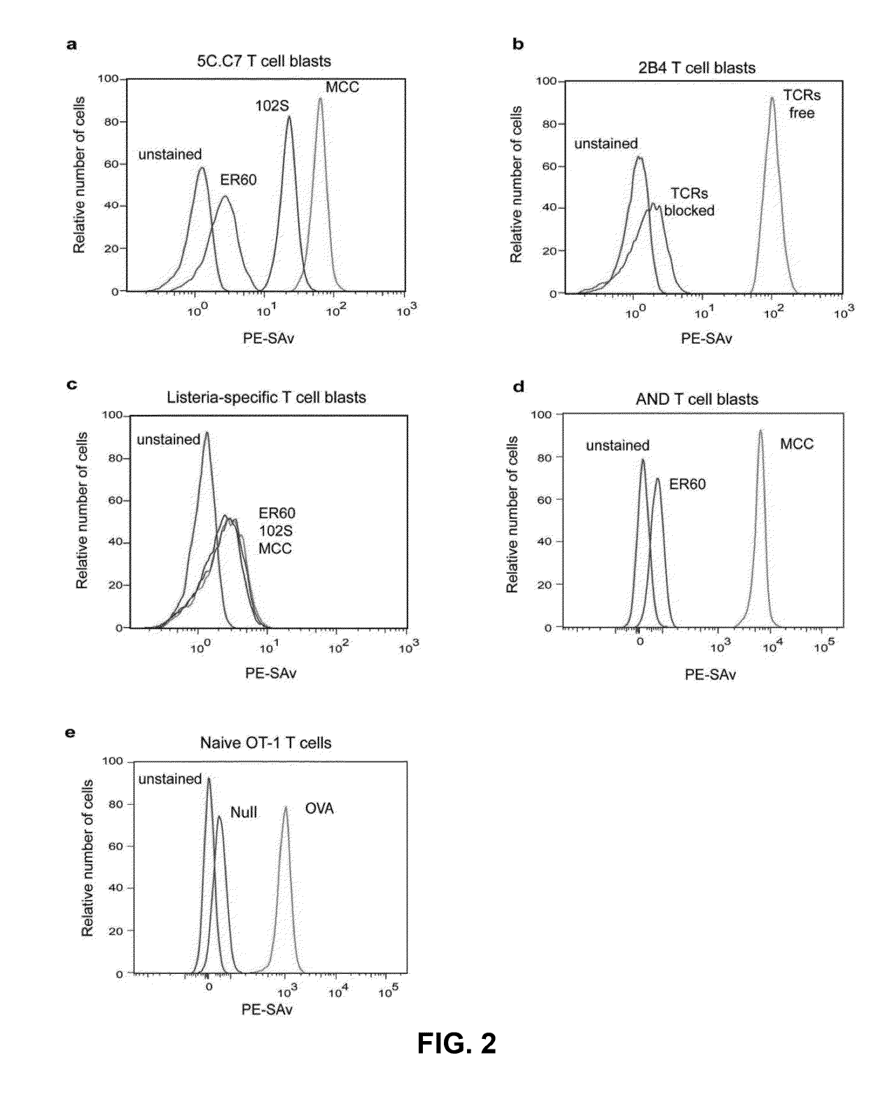

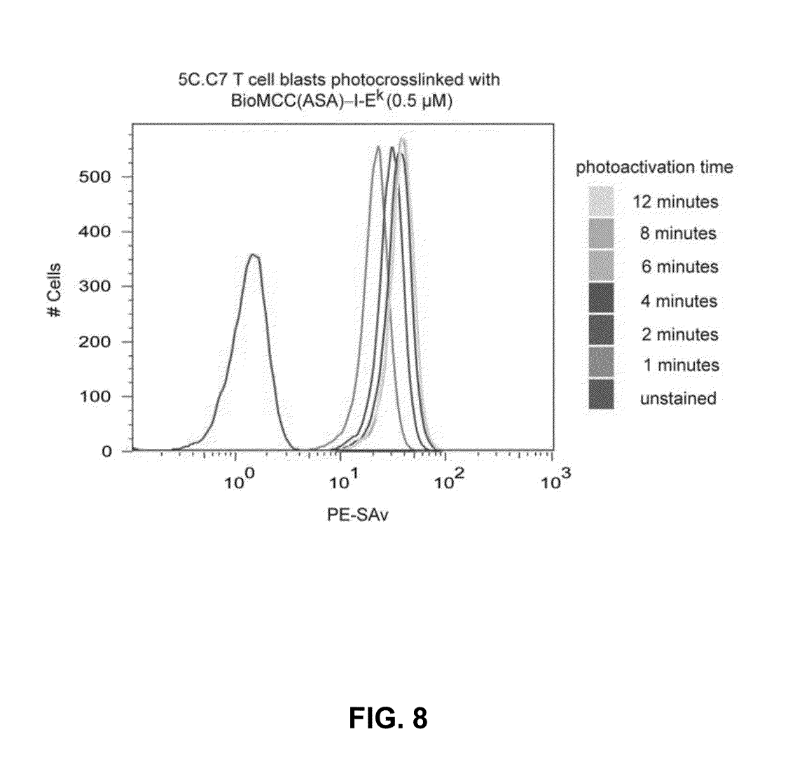

The photocrosslinkable pMHC monomers capability of staining T cell blasts extracted from 5C.C7 TCR transgenic mice was investigated. Cells were incubated with pMHC ligands (1 μM) on ice for 30 minutes, and then the photocrosslinking reaction was induced by UV irradiation (5×15 watt bulbs, 365 nm, 5 minutes). The cells were washed extensively to remove unbound pMHC molecules, and PE-conjugated streptavidin (PE-SAv) added to label cells via the biotin moiety of bound pMHCs. Subsequent flow cytometric analysis showed that BioMCC(ASA)-I-Ek was the most efficient among these three pMHCs in staining 5C.C7 T cells, Bio102S(ASA)-I-Ek was less efficient, and BioER60(ASA)-I-Ek stained T cells least efficiently (FIG. 2a). The varied staining efficiency was consistent with their different affinities to the 5C.C7 TCR. To verify that the photocrosslinking was specific to cognate TCRs, an anti-TCR antibody was used to block the TCR prior to UV irradiation, and it was found that the staining signal from BioMCC(ASA)-I-Ek was completely abrogated (FIG. 2b). In another experiment, the three pMHCs were used to stain a Listeria-specific T-cell clone that recognizes the LLO190-201 peptide binding to I-Ab, and it was found that none could stain these T cells significantly (FIG. 2c). Taken together, these results show that a photocrosslinkable, monomeric pMHC can be used to stain its cognate T cell with excellent specificity. The 5-minute UV exposure time was used because this is where labeling reached a plateau (FIG. 8).

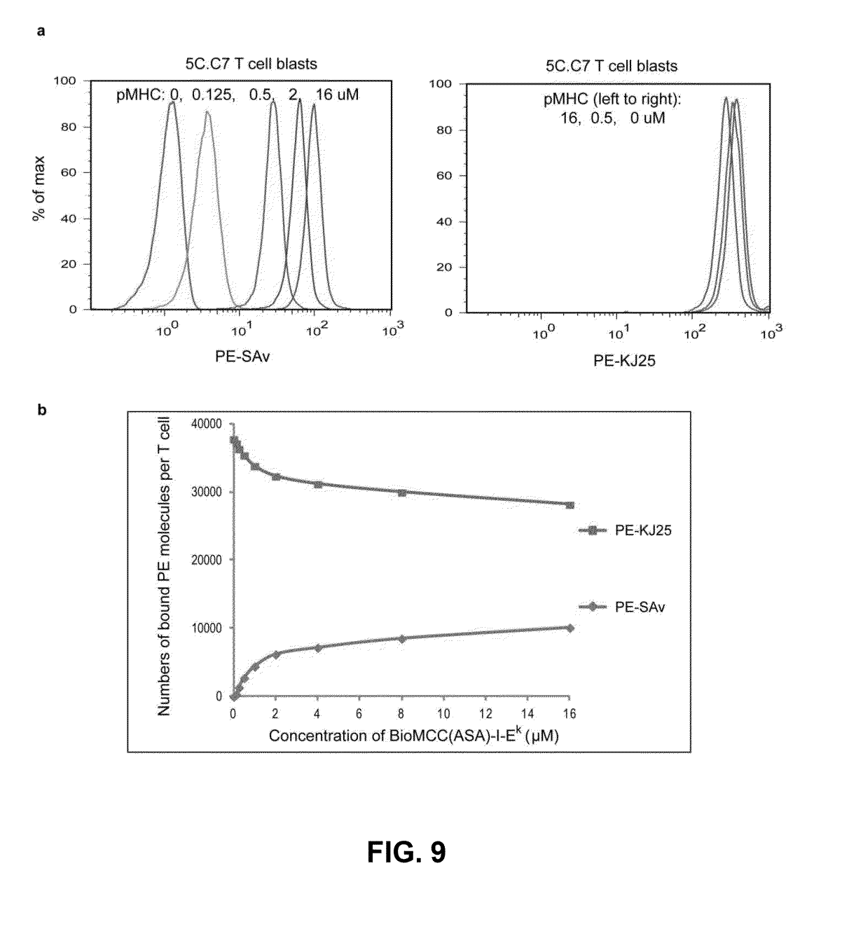

To characterize the efficiency of photocrosslinking, the number of crosslinked pMHC molecules per T cell was determined. 5C.C7 T cell blasts were photocrosslinked with a series of 2-fold-diluted BioMCC(ASA)-I-Ek (from 0.125 to 16 μM), and then the bound pMHC molecules were labeled using an excess of PE-SAv. Next, flow cytometry was used to measure the average fluorescence intensity of T cells and of beads conjugated with known numbers of PE molecules, from which the number of PE molecules per cell was calculated (Table 1 and FIG. 9). For example, it was found that the use of 2 μM BioMCC(ASA)-I-Ek led to an average of 6,194 permanently bound pMHC molecules per cell. That was about 16% of the number of total TCRs on a T cell surface, which was estimated to be 38,000 per T cell based on the binding characteristics of a PE-conjugated anti-TCR antibody (Table 1).

TABLE 1
Numbers of bound PE-SAv and PE-KJ25 molecules per T cell from
5C.C7 T cell blasts photocrosslinked with BioMCC(ASA)-l-Ek on ice.

| BioMCC(ASA)-l-E^{k} | Number of bound | Number of bound |
|---|---|---|
| (μM) | PE-SAv per T cell | PE-KJ25 per T cell |
| 0 | 0 | 37,760 |
| 0.125 | 304 | 37,144 |
| 0.25 | 1,354 | 36,324 |
| 0.5 | 2,738 | 35,387 |
| 1 | 4,417 | 33,816 |
| 2 | 6,194 | 32,309 |
| 4 | 7,177 | 31,169 |
| 8 | 8,508 | 29,967 |
| 16 | 10,101 | 28,152 |

The accessibility of TCR variable regions was examined after T cells were photocrosslinked with pMHCs. To do so, a PE-conjugated anti-TCR Vβ3 antibody KJ25 (PE-KJ25) was used to stain 5C.C7 T cell blasts crosslinked with BioMCC(ASA)-I-Ek, and the number of bound PE-KJ25 per cell was determined using the same method as described above. It was found that the number of bound PE-KJ25 molecules was inversely correlated with that of bound PE-SAv (Table 1 and FIG. 9), which indicates that TCR-pMHC crosslinking and TCR-KJ25 association are mutually exclusive. This is to be expected as part of the KJ25 epitope is in the CDR2 region of Vβ3 27. Based on this discovery, fluorescently-labeled KJ25 was used to monitor the dynamics of unengaged TCRs on T-cell surfaces.

Comparing Photocrosslinkable pMHC Monomers with pMHC Tetramers for T-Cell Staining.

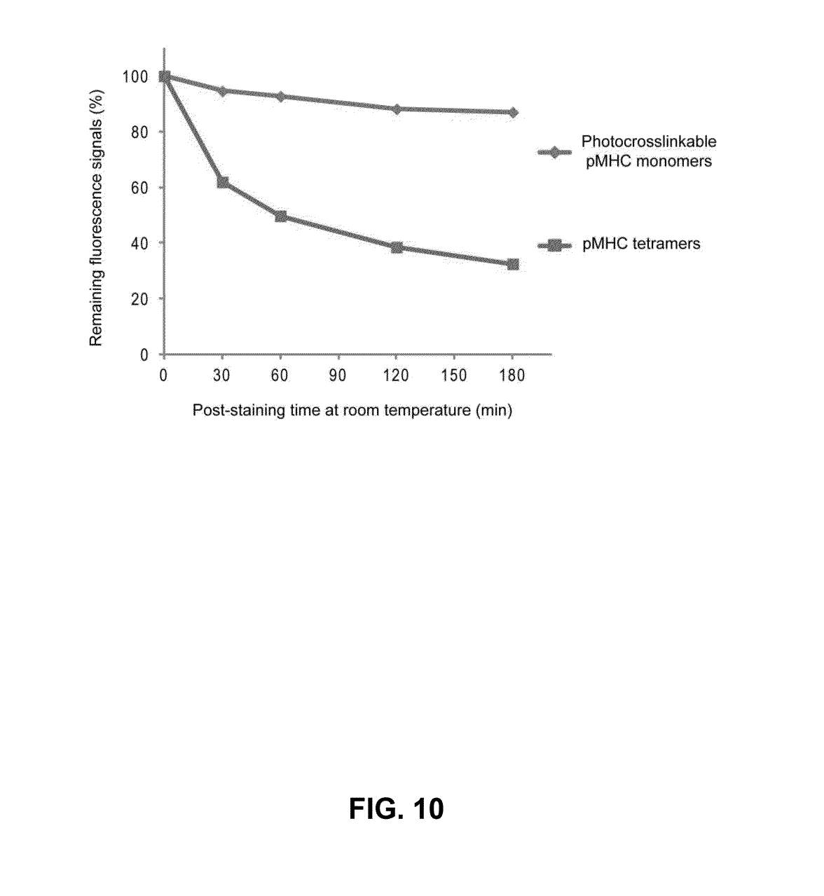

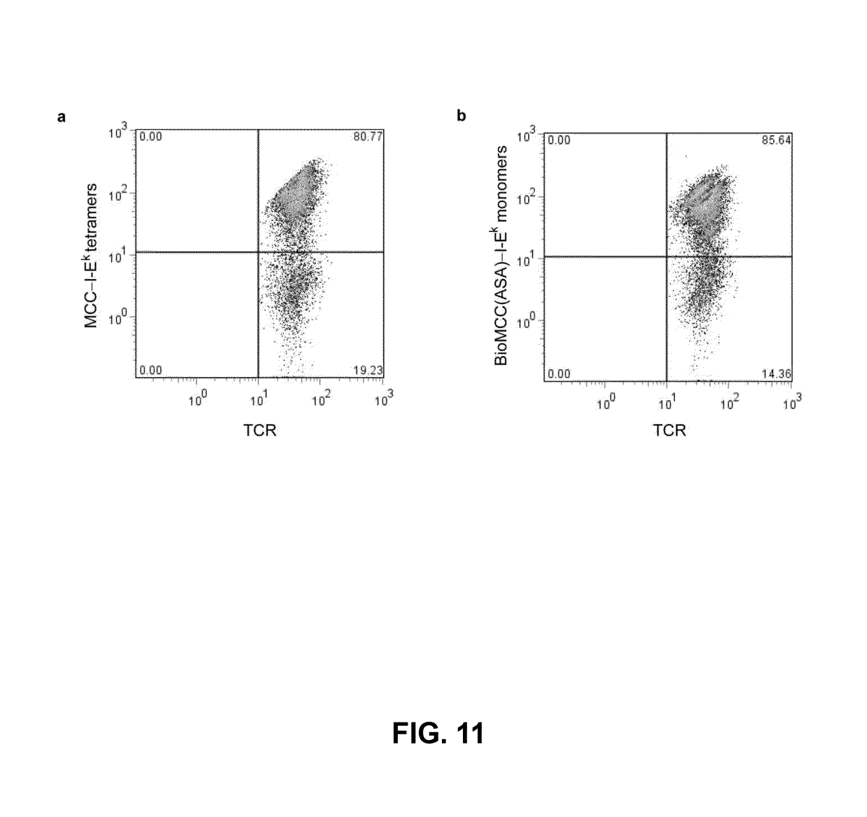

Both the PE-conjugated MCC-I-Ek tetramer and the photocrosslinkable BioMCC(ASA)-I-Ek monomer (together with PE-SAv) were used to stain 5C.C7 T cell blasts. They both efficiently stained these T cells. However, after one hour at room temperature after staining, the signal from the tetramer decreased by approximately 50%, while that of the photocrosslinkable monomer declined only about 5% (FIG. 10). This difference is consistent with the fact that multivalent pMHC-TCR interactions are still reversible, while pMHC-TCR crosslinking is irreversible. The two reagents were also used to analyze a day 8 culture of 5C.C7 TCR β chain transgenic T cell blasts that were primed with the MCC peptide, which consisted of a repertoire of T cells that recognize MCC-I-Ek with differing affinities. While both were capable of detecting cognate T cells in a mixture, the photocrosslinkable pMHC monomer had a slightly better resolution in discerning multiple T-cell populations with varied affinities (FIG. 11).

Using Photocrosslinkable pMHC Monomers to Label Other T Cells.

To test the generality of this method on other T cells specific for MCC, 2B4 T cells (FIG. 2b) and a mixture of T cell clones prepared from 5C.C7 TCR β chain transgenic mice and primed with the MCC peptide (FIG. 11) were successfully labeled. T cell blasts from a third widely used MCC-I-Ek specific TCR transgenic mouse, AND, which has a very different fine specificity than either 5C.C7 and 2B4 28-29, were stained efficiently (FIG. 2d). These TCRs have distinct Vα chain sequences and these lie over the amino terminus of the MCC peptide bound by I-Ek, yet all are effectively captured by the photocrosslinker. These results show that this approach is general for detecting many other antigen-specific, class II restricted T cells.

The use of this method to detect class I restricted TCRs was investigated. OT-1 TCR was selected which recognize the OVA257-264 peptide (SIINFEKL) binding to the murine MHC class I H-2 Kb molecule (OVA-Kb) (Carbone, F. R. & Bevan, M. J. Induction of ovalbumin-specific cytotoxic T cells by in vivo peptide immunization. J Exp Med 169, 603-612 (1989)). First, a photocrosslinkable OVA257-264 peptide was synthesized. Unlike the class II pMHC whose peptide binding groove is open at both ends, both termini of the OVA257-264 peptide are embedded in the peptide-binding groove of H-2 Kb (Wilson et al., Crystal structure of an H-2 Kb-ovalbumin peptide complex reveals the interplay of primary and secondary anchor positions in the major histocompatibility complex binding groove. Proc Natl Acad Sci USA 92, 2479-2483 (1995)). The C-terminus of OVA257-264 is extendable (Davis et al., T cell killing does not require the formation of a stable mature immunological synapse. Nat Immunol 5, 524-530 (2004)). Accordingly, a derivative of OVA257-264 peptide was synthesized containing an extended carboxyl-terminal sequence (SIINFEKLGGGGSGCS), and then ASA attached to the cysteine residue to produce a photocrosslinkable OVA257-264 peptide—termed as OVA(ASA). OVA(ASA) was loaded to a biotinylated H-2 Kb utilizing a UV-mediated peptide exchange method described by Toebes, M., et al. (Design and use of conditional MHC class I ligands. Nat Med 12, 246-251 (2006)). To do so, in vitro refolding was used to produce an exchangeable pMHC complex consisting of H-2 Kb and a photocleavable peptide (OVA263ANP, SIINFE[ANP]L). It was then UV irradiated to generate empty H-2 Kb molecules (Stratalinker 2400, 5×15 watt bulbs, 365 nm, 10 minutes), followed by adding OVA(ASA) in dark to produce OVA(ASA)-Kb. In parallel, a photocrosslinkable derivative of a null peptide (SIAAFASL) binding to H-2 Kb as a negative control, termed as SIAAFASL(ASA)-Kb, was produced.

Next, naïve OT-1 T cells from the OT-1/Rag1 KO mouse were isolated, and cell aliquots were mixed with OVA(ASA)-Kb (1 μM) and SIAAFASL(ASA)-Kb (1 μM), respectively. Cells were UV irradiated using the same protocol as described above, cells were washed extensively to remove unbound pMHC molecules, and then PE-SAv was added to label cells via the biotin moiety of bound pMHCs. Flow cytometric analysis showed that the OVA(ASA)-Kb monomer was more than 10 times more efficient in staining OT-1 T cells than the null SIAAFASL(ASA)-Kb monomer (FIG. 2e). Thus, the photocrosslinkable pMHC monomer approach can also be used to detect class I restricted T cells.

Activating T Cells Using Photocrosslinkable pMHCs.

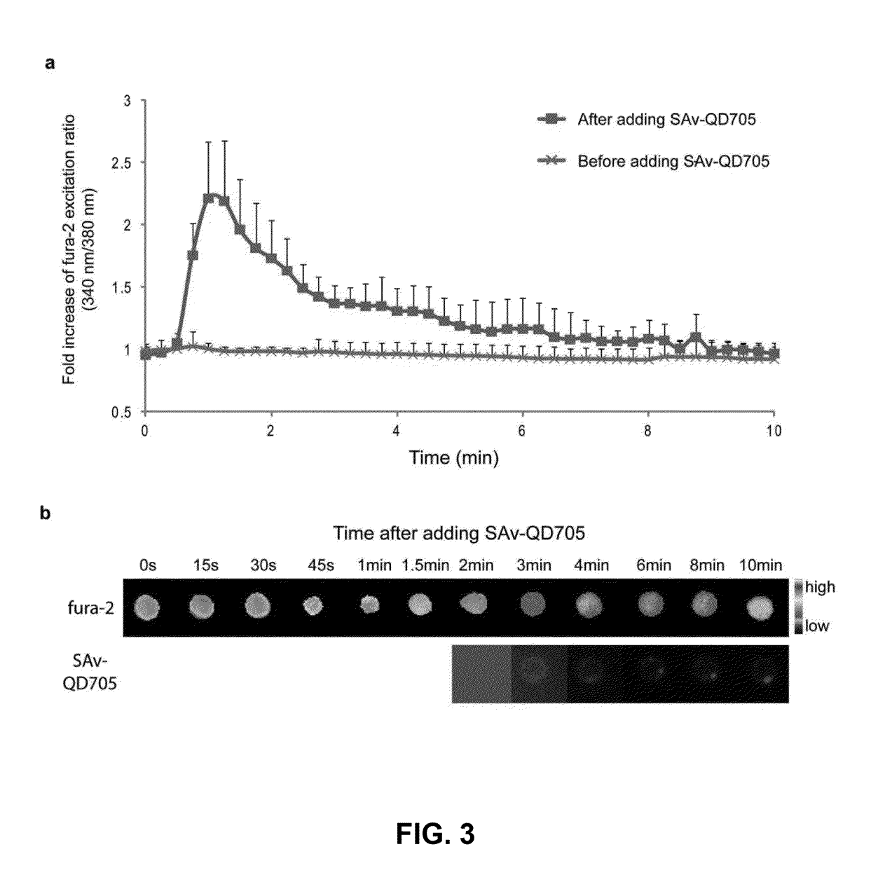

The utility of these reagents in dissecting T-cell activation was tested. It was first assessed whether crosslinked pMHC monomers could stimulate T cells, using calcium imaging. 5C.C7 T cell blasts were loaded with the calcium indicator dye fura-2 prior to photocrosslinking with BioMCC(ASA)-I-Ek (2 μM). After photocrosslinking, cells were washed to remove unbound pMHC ligands, and the intracellular calcium concentration of T cells at 37° C. monitored using time-lapse, 3D fluorescence microscopy. Although a large fraction of TCRs were occupied by photocrosslinked pMHC molecules (more than 6,000 TCRs per cell, or about 16% of total TCRs, see Table 1), no elevation of cytoplasmic calcium (FIG. 3a) was observed. The T cells did flux calcium after streptavidin-coated quantum dots 705 (SAv-QD705, 10 nm, 6-8 SAv per QD, Invitrogen) were added to aggregate pMHC-TCR complexes. Continuous imaging of QD showed that multimerized pMHC-TCR complexes continued to move together and formed a large cluster (FIG. 3b). These results show that monomeric pMHCs crosslinking to TCRs are not stimulatory, but that they are after subsequent aggregation.

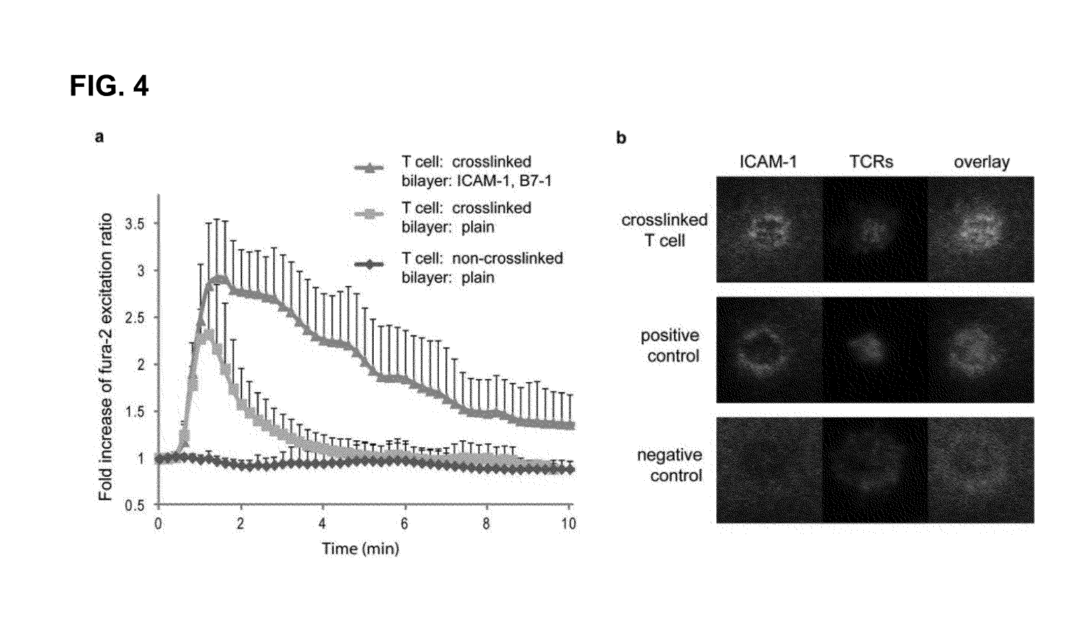

The activation ability of these permanently engaged pMHC ligands using a glass-supported planar lipid bilayer as a surrogate APC surface was examined. Since the planar lipid bilayer was seeded with 10% nickel nitrilotriacetic acid (Ni-NTA) lipids, and the bound pMHC ligands on T-cell surfaces contained a His6-tagged carboxyl tail in both α and β subunits, this would have the effect of causing T cells to adhere to the bilayer via the His6/Ni-NTA conjugation and possibly activate. It was observed that T cells fluxed calcium shortly after landing onto lipid bilayers. The induced calcium flux lasted about three minutes in the absence of ICAM-1 adhesion molecules and B7-1 costimulatory molecules on the lipid bilayer, and lasted more than 10 minutes in their presence (FIG. 4a). It was also tested whether these T cells could form immunological synapses. Alexa-647 conjugated single-chain variable fragments (scFv) of anti-TCR-Cβ antibody H57 (H57-scFv-A647) was used to label TCRs prior to loading T cells onto a planar lipid bilayer, which presents B7-1 and Alexa-555 conjugated ICAM-1 (ICAM-1-A555). Live-cell fluorescent imaging showed that TCRs on the T-cell surface were partially accumulated in an area that was surrounded by a ring of ICAM-1 molecules (FIG. 4b), indicating the formation of immunological synapses. These results show that covalently bound pMHC ligands on planar lipid bilayers are stimulatory. Since the same ligand is not stimulatory in solution, activation depends on aggregating TCRs and/or mechanical forces.

A Covalently Bound pMHC Ligand is More Stimulatory than an Agonist pMHC.

Next, the stimulatory potency of photocrosslinked pMHC ligands and standard agonist pMHC ligands without crosslinking was compared. This requires a determination of the number of each kind of pMHC molecules used to stimulate T cells. Since pMHC molecules accumulate in the cSMAC after engaging TCRs12, the comparison was made based on the number of synaptic pMHC molecules. A day 7 culture of 5C.C7 T cells was divided into two aliquots: one was photocrosslinked with BioMCC(ASA)-I-Ek ligands, while the other was exposed to the same amount of UV light but without pMHC ligands, and then used for subsequent activation with the standard MCC-I-Ek ligand. Both samples were then further divided into two new aliquots. The first aliquot was used for the quantification of synaptic pMHC molecules and the second aliquot for functional assays, including calcium flux and cytokine secretion.

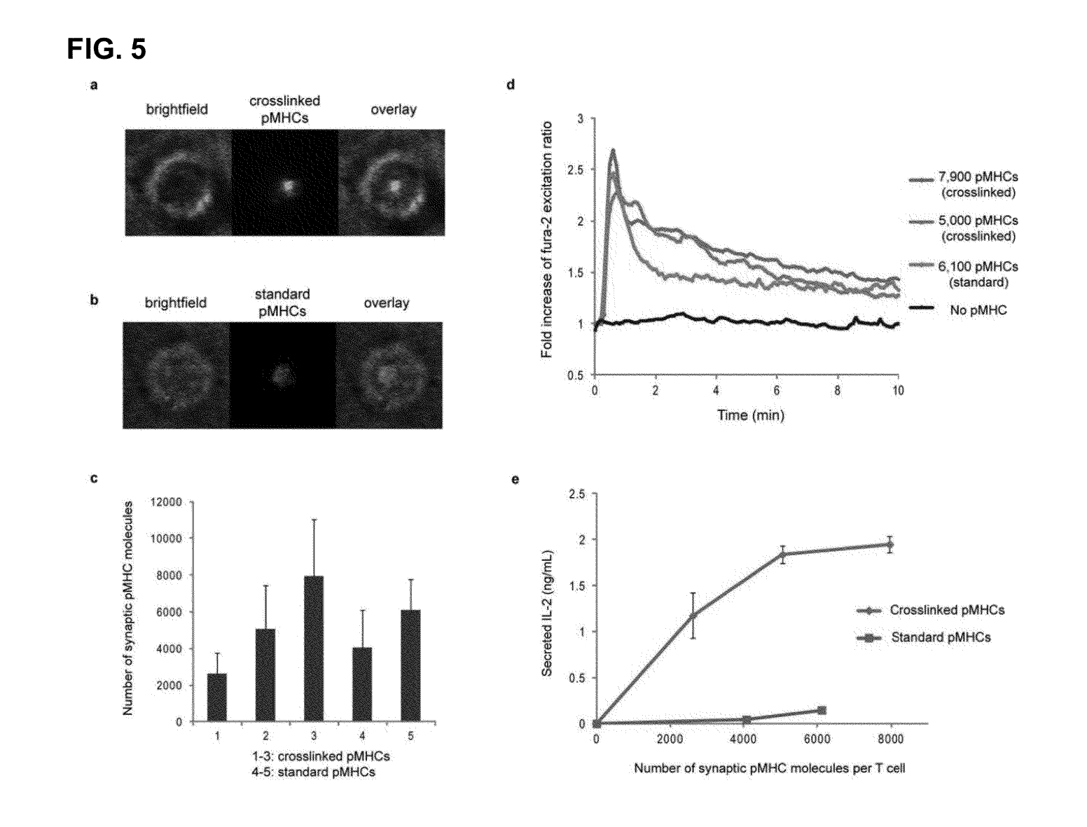

To calculate the number of synaptic pMHC molecules, a fluorescence single-molecule counting assay was used. For T cells crosslinked with BioMCC(ASA)-I-Ek, Alexa-555 conjugates of monovalent-streptavidin (monoSAv-A555) were used to label the associated pMHC molecules on T-cell surfaces. Most BioMCC(ASA)-I-Ek molecules (in conjugation with TCRs) were accumulated in the cSMAC after the T cell had contacted the planar lipid bilayers presenting ICAM-1 and B7-1 (FIG. 5a). The average fluorescence intensity of the cSMAC regions was measured, as well as that of a single Alexa-555 molecule from a separate bilayer presenting an Alexa-555 labeled pMHC at low density. Based on these measurements, the number of synaptic pMHC molecules was calculated. For T cells that were not photocrosslinked with pMHCs, they were activated using a lipid bilayer presenting ICAM-1, B7-1, and a standard pMHC molecule MCC(Cy5)-I-Ek with a Cy5 dye conjugated to the C-terminus of MCC peptide 12. Fluorescent imaging showed that MCC-I-Ek ligands were also accumulated in the cSMAC after T-cell activation (FIG. 5b). The number of synaptic MCC-I-Ek molecules were calculated using the same method as described above. To make a comparison between the stimulatory potency of a photocrosslinked MCC-I-Ek and that of a standard ligand, those T cell samples that had similar density of synaptic pMHC ligands were selected for subsequent functional assays (FIG. 5c).

The elevation of cytoplasmic calcium between T cells activated by the standard agonist pMHCs (6100 per cell) was compared with T cells activated by similar amounts of photocrosslinked pMHCs (5100 or 7900 per cell). Time-lapse calcium imaging showed that both ligands were capable of inducing a sharp increase of fura-2 excitation ratio (340 nm/380 nm) in T cells, which reached its peak value (about 2.5 fold above the baseline) in less than 45 seconds (FIG. 5d). However, the elevated calcium signal induced by the photocrosslinked pMHCs was maintained over a significantly longer time than that induced by the standard agonist pMHC. Further, the T cells were cultured overnight and interleukin-2 (IL-2) secretion measured by ELISA. T cells activated by the photocrosslinked pMHCs (2600, 5100, or 7900 per cell) produced more IL-2 than those activated by standard pMHC ligands (4100 or 6100 per cell) (FIG. 5e). Taken together, these results show that on a molar basis and at high densities, the permanently engaged pMHC ligand is more stimulatory than the conventional agonist ligand.

Engaged TCRs, but not Unengaged Ones, Rapidly Move to the cSMAC after Activation.

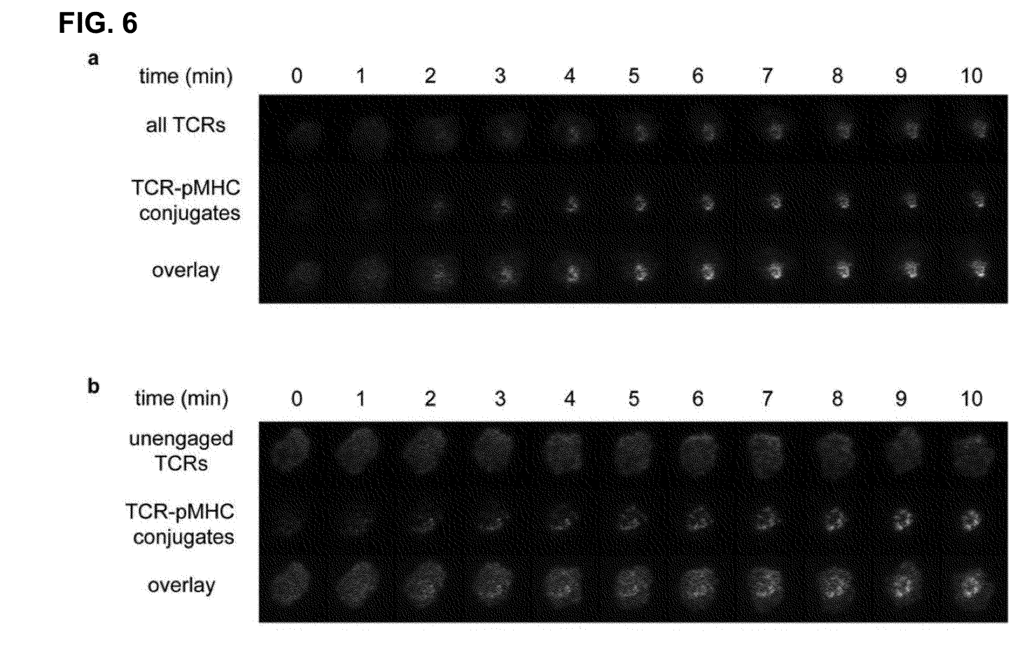

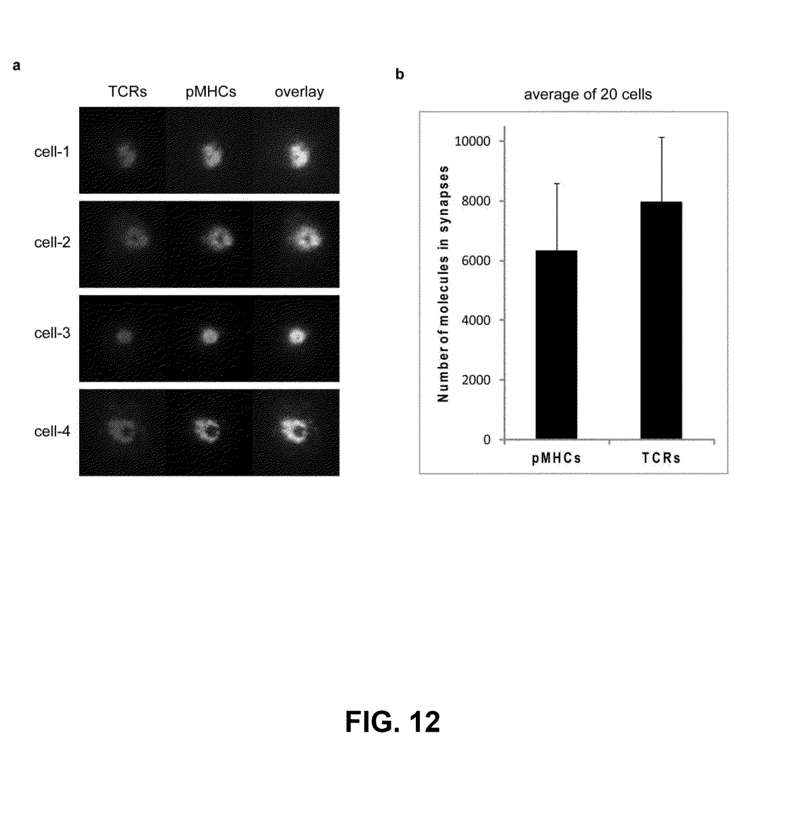

Next, this reagent was used to probe synapse formation and TCR dynamics in T-cell activation. Both monoSAv-A555 and H57-scFv-A647 were used to label T cells photocrosslinked with BioMCC(ASA)-I-Ek, which allowed monitoring of the dynamics of pMHC and TCR molecules simultaneously using video fluorescence microscopy. It was found that both of them migrated to the cSMAC within just a few minutes after T cells landed onto planar lipid bilayers (FIG. 6a). Meanwhile, there were many TCRs visible in the peripheral area of the T-cell surface, which were mostly unengaged (FIG. 4b and FIG. 6a). To test whether some of the unengaged TCRs had migrated concurrently to the cSMAC with the engaged ones, the numbers of synaptic pMHCs and TCRs in a photocrosslinked T cell were determined. The same fluorescent single-molecule counting method described above was used to calculate the average number of synaptic TCRs and pMHCs. It was found that TCRs (˜7900 per cSMAC) were in a slight excess over pMHCs (˜6100 per cSMAC) in the synapse (FIG. 12), suggesting that most synaptic TCRs were engaged with pMHCs.

Results described above indicated that TCR-pMHC crosslinking and TCR-KJ25 association are mutually exclusive (Table 1 and FIG. 9). To further verify that unengaged TCRs had not accumulated in the cSMAC, an Fab′ of the anti-Vβ3 antibody KJ25 conjugated with Alexa-647 (KJ25-Fab-A647) was used to label unengaged TCRs on 5C.C7 T cells that were photocrosslinked with BioMCC(ASA)-I-Ek. Subsequent video fluorescent imaging showed that unengaged TCRs did not move into the cSMAC to any noticeable extent, but rather remained evenly distributed all over the T cell surface (FIG. 6b). Taken together, these results show that engaged TCRs are preferentially transported to the cSMAC in the initial phases of T-cell activation, whereas unengaged TCRs are not.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of labeling a T cell according to specificity of an antigen T cell receptor (TCR), the method comprising:
contacting a T cell with a monomeric MHC-peptide complex comprising a peptide, wherein the peptide comprises:
a core TCR-recognition region; and
a photocrosslinking moiety that is located outside of the core TCR-recognition region of the peptide;
under conditions by which the TCR of the T cell and the core TCR-recognition region specifically bind; and
applying a stimulus to crosslink the MHC-peptide complex to the T cell.

2. The method of claim 1, wherein the photocrosslinking moiety is connected to a region of the peptide adjacent to the core TCR-recognition region.

3. The method of claim 1, wherein the peptide is not crosslinked to the MHC of the MHC-peptide complex.

4. The method of claim 1, wherein the core TCR-recognition region of the MHC-peptide complex consists of natural underivatized amino acids.

5. The method of claim 4, wherein the photocrosslinking moiety is connected to an amino acid sidechain of the peptide.

6. The method of claim 4, wherein the photocrosslinking moiety is connected to a terminus of the peptide.

7. The method of claim 1 wherein the stimulus for crosslinking is radiation.

8. The method of claim 7 wherein the radiation is UV light.

9. The method of claim 8, wherein the peptide of the MHC-peptide complex is described by formula (II): 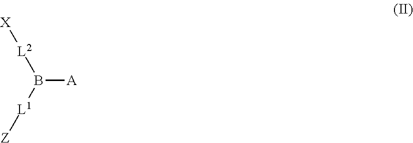
wherein:
A is the core TCR-recognition region;
X is the photocrosslinking moiety;
L¹ and L² are optional non-peptidic linkers;
B is a peptidic linker comprising one or more amino acid residues; and
Z is an optional detectable moiety or an optional crosslinking moiety.

10. The method of claim 9, wherein the peptide of the MHC-peptide complex is described by formula (III): 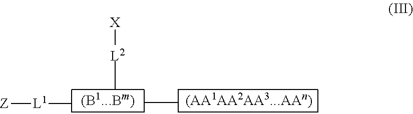
wherein:
X, Z, L¹ and L² are as described for Formula (II);
(AA¹ AA² AA³ . . . AAⁿ) is a sequence of n amino acid residues from AA¹ to AAⁿ that comprises the core TCR-recognition element, wherein n is an integer from 5 to 12;
(B¹ . . . B^{m}) is a sequence of m residues from B¹ to B^{m}, wherein m is an integer from 2 to 12, and residue B^{m} is N-terminal to the TCR-recognition element;
X is attached to the peptide via the B^{m}, B^{(m-1)} or B^{(m-2)} residue sidechain.

11. A method for detecting a T cell in a sample according to specificity of an antigen T cell receptor (TCR), the method comprising:
contacting the sample with a monomeric MHC-peptide complex comprising a peptide, wherein the peptide comprises a core TCR-recognition region and a photocrosslinking moiety, under conditions by which the TCR of the T cell and the core TCR-recognition region specifically bind; and
applying a light stimulus to crosslink the MHC-peptide complex to the T cell to produce a crosslinked T cell; and
detecting the crosslinked T cell.

12. The method of claim 11, further comprising separating the crosslinked T cell from non-crosslinked T cells.

13. The method of claim 12, wherein the peptide further comprises a detectable label and the separating is performed on the basis of the detectable label.

14. A monomeric MHC-peptide complex for crosslinking to a T cell according to specificity of an antigen T cell receptor (TCR), wherein the MHC-peptide complex is described by formula (I):
α-β-P (I)
wherein:
α comprises a soluble form of an α-chain of a class I or class II MHC protein;
β comprises a soluble form of (i) a β-chain of a class II MHC protein, or (ii) β₂ microglobulin for a class I MHC protein; and
P is a peptide comprising a core TCR-recognition region and a photocrosslinking moiety attached to the peptide at a region outside of the core TCR-recognition region;
and wherein P is bound in the groove formed by two membrane distal domains of (i) the α-chain for a class I MHC protein or (ii) α-chain and the β-chains for a class II MHC protein.

15. The method of claim 14, wherein the photocrosslinkable peptide reagent is described by formula (II): 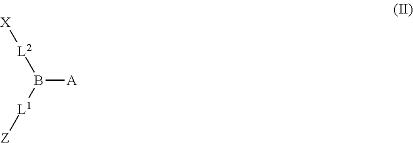
wherein:
A is the core TCR-recognition region;
X is the photocrosslinking moiety;
L¹ and L² are optional non-peptidic linkers;
B is a peptidic linker comprising one or more amino acid residues; and
Z is an optional detectable moiety or an optional crosslinking moiety.

16. A kit comprising:
a photocrosslinkable peptide reagent comprising a core TCR-recognition region and a photocrosslinking moiety attached to the peptide reagent at a region outside of the core TCR-recognition region;
one or more components selected from: one or more MHC components, one or more T cells; a light source; a buffer and instructions for use.

17. The kit of claim 16, wherein the photocrosslinkable peptide reagent is described by formula (II): 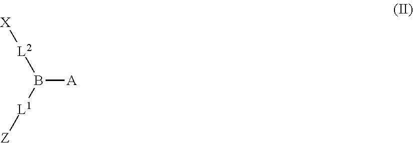
wherein:
A is the core TCR-recognition region;
X is the photocrosslinking moiety;
L¹ and L² are optional non-peptidic linkers;
B is a peptidic linker comprising one or more amino acid residues; and
Z is an optional detectable moiety or an optional crosslinking moiety.
